(19) United States
(12) Patent Application Publication
(10) Pub. No.: US 2006/0247263 A1
(43) Pub. Date: Nov. 2, 2006

(54) Substituted heterocyclic compounds and methods of use

(75) Inventor: Aaron C. Siegmund, Ventura, CA (US)

Correspondence Address: AMGEN INC., MAIL STOP 28-2-C, ONE AMGEN CENTER DRIVE, THOUSAND OAKS, CA, 91320-1799, US

(73) Assignee: Amgen Inc., Thousand Oaks, CA (US)

(21) Appl. No.: 11/400,446

(22) Filed: Apr. 6, 2006

Related U.S. Application Data
(60) Provisional application No. 60/673,103, filed on Apr. 19, 2005.

(51) Int. Cl. A61K 31/506; C07D 403/02

(52) U.S. Cl. 514/275; 544/329

(57) ABSTRACT

The present invention relates to compounds having the general structure: 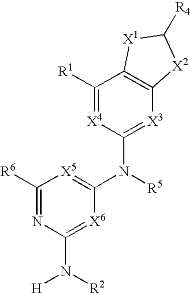 and pharmaceutically acceptable salts and hydrates thereof. Also included is a method of treatment of inflammation, rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount a compound as described above.

This application claims the benefit of U.S. Provisional Application No. 60/673,103 filed Apr. 19, 2005, which is hereby incorporated by reference.

### BACKGROUND OF THE INVENTION

The present invention comprises a new class of compounds useful in treating diseases, such as TNF-α, IL-1β, IL-6 and/or IL-8 mediated diseases and other maladies, such as pain and diabetes. In particular, the compounds of the invention are useful for the prophylaxis and treatment of diseases or conditions involving inflammation. This invention also relates to intermediates and processes useful in the preparation of such compounds.

Interleukin-1 (IL-1) and Tumor Necrosis Factor α (TNF-α) are pro-inflammatory cytokines secreted by a variety of cells, including monocytes and macrophages, in response to many inflammatory stimuli (e.g., lipopolysaccharide—LPS) or external cellular stress (e.g., osmotic shock and peroxide).

Elevated levels of TNF-α and/or IL-1 over basal levels have been implicated in mediating or exacerbating a number of disease states including rheumatoid arthritis; Pagets disease; osteoporosis; multiple myeloma; uveititis; acute and chronic myelogenous leukemia; pancreatic β cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; Reiter's syndrome; type I and type II diabetes; bone resorption diseases; graft vs. host reaction; ischemia reperfusion injury; atherosclerosis; brain trauma; multiple sclerosis; cerebral malaria; sepsis; septic shock; toxic shock syndrome; fever, and myalgias due to infection. HIV-1, HIV-2, HIV-3, cytomegalovirus (CMV), influenza, adenovirus, the herpes viruses (including HSV-1, HSV-2), and herpes zoster are also exacerbated by TNF-α.

It has been reported that TNF-α plays a role in head trauma, stroke, and ischemia. For instance, in animal models of head trauma (rat), TNF-α levels increased in the contused hemisphere (Shohami et al., J. Cereb. Blood Flow Metab. 14, 615 (1994)). In a rat model of ischemia wherein the middle cerebral artery was occluded, the levels of TNF-α mRNA of TNF-α increased (Feurstein et al., Neurosci. Lett. 164, 125 (1993)). Administration of TNF-α into the rat cortex has been reported to result in significant neutrophil accumulation in capillaries and adherence in small blood vessels. TNF-α promotes the infiltration of other cytokines (IL-1β, IL-6) and also chemokines, which promote neutrophil infiltration into the infarct area (Feurstein, Stroke 25, 1481 (1994)). TNF-α has also been implicated to play a role in type II diabetes (Endocrinol. 130, 43-52, 1994; and Endocrinol. 136, 1474-1481, 1995).

TNF-α appears to play a role in promoting certain viral life cycles and disease states associated with them. For instance, TNF-α secreted by monocytes induced elevated levels of HIV expression in a chronically infected T cell clone (Clouse et al., J. Immunol. 142, 431 (1989)). Lahdevirta et al., (Am. J. Med. 85, 289 (1988)) discussed the role of TNF-α in the HIV associated states of cachexia and muscle degradation.

TNF-α is upstream in the cytokine cascade of inflammation. As a result, elevated levels of TNF-α may lead to elevated levels of other inflammatory and proinflammatory cytokines, such as IL-1, IL-6, and IL-8.

Elevated levels of IL-1 over basal levels have been implicated in mediating or exacerbating a number of disease states including rheumatoid arthritis; osteoarthritis; rheumatoid spondylitis; gouty arthritis; inflammatory bowel disease; adult respiratory distress syndrome (ARDS); psoriasis; Crohn's disease; ulcerative colitis; anaphylaxis; muscle degeneration; cachexia; Reiter's syndrome; type I and type II diabetes; bone resorption diseases; ischemia reperfusion injury; atherosclerosis; brain trauma; multiple sclerosis; sepsis; septic shock; and toxic shock syndrome. Viruses sensitive to TNF-α inhibition, e.g., HIV-1, HIV-2, HIV-3, are also affected by IL-1.

TNF-α and IL-1 appear to play a role in pancreatic β cell destruction and diabetes. Pancreatic β cells produce insulin which helps mediate blood glucose homeostasis. Deterioration of pancreatic β cells often accompanies type I diabetes. Pancreatic β cell functional abnormalities may occur in patients with type II diabetes. Type II diabetes is characterized by a functional resistance to insulin. Further, type II diabetes is also often accompanied by elevated levels of plasma glucagon and increased rates of hepatic glucose production. Glucagon is a regulatory hormone that attenuates liver gluconeogenesis inhibition by insulin. Glucagon receptors have been found in the liver, kidney and adipose tissue. Thus glucagon antagonists are useful for attenuating plasma glucose levels (WO 97/16442, incorporated herein by reference in its entirety). By antagonizing the glucagon receptors, it is thought that insulin responsiveness in the liver will improve, thereby decreasing gluconeogenesis and lowering the rate of hepatic glucose production.

In rheumatoid arthritis models in animals, multiple intra-articular injections of IL-1 have led to an acute and destructive form of arthritis (Chandrasekhar et al., Clinical Immunol Immunopathol. 55, 382 (1990)). In studies using cultured rheumatoid synovial cells, IL-1 is a more potent inducer of stromelysin than is TNF-α (Firestein, Am. J. Pathol. 140, 1309 (1992)). At sites of local injection, neutrophil, lymphocyte, and monocyte emigration has been observed. The emigration is attributed to the induction of chemokines (e.g., IL-8), and the up-regulation of adhesion molecules (Dinarello, Eur. Cytokine Netw. 5, 517-531 (1994)).

IL-1 also appears to play a role in promoting certain viral life cycles. For example, cytokine-induced increase of HIV expression in a chronically infected macrophage line has been associated with a concomitant and selective increase in IL-1 production (Folks et al., J. Immunol. 136, 40 (1986)). Beutler et al. (J. Immunol. 135, 3969 (1985)) discussed the role of IL-1 in cachexia. Baracos et al. (New Eng. J. Med. 308, 553 (1983)) discussed the role of IL-1 in muscle degeneration.

In rheumatoid arthritis, both IL-1 and TNF-α induce synoviocytes and chondrocytes to produce collagenase and neutral proteases, which leads to tissue destruction within the arthritic joints. In a model of arthritis (collagen-induced arthritis (CIA) in rats and mice), intra-articular administration of TNF-α either prior to or after the induction of CIA led to an accelerated onset of arthritis and a more severe course of the disease (Brahn et al., Lymphokine Cytokine Res. 11, 253 (1992); and Cooper, Clin. Exp. Immunol. 898, 244 (1992)).

IL-8 has been implicated in exacerbating and/or causing many disease states in which massive neutrophil infiltration into sites of inflammation or injury (e.g., ischemia) is mediated by the chemotactic nature of IL-8, including, but not limited to, the following: asthma, inflammatory bowel disease, psoriasis, adult respiratory distress syndrome, cardiac and renal reperfusion injury, thrombosis and glomerulonephritis. In addition to the chemotaxis effect on neutrophils, IL-8 also has the ability to activate neutrophils. Thus, reduction in IL-8 levels may lead to diminished neutrophil infiltration.

Several approaches have been taken to block the effect of TNF-α. One approach involves using soluble receptors for TNF-α (e.g., TNFR-55 or TNFR-75), which have demonstrated efficacy in animal models of TNF-α-mediated disease states. A second approach to neutralizing TNF-α using a monoclonal antibody specific to TNF-α, cA2, has demonstrated improvement in swollen joint count in a Phase II human trial of rheumatoid arthritis (Feldmann et al., Immunological Reviews, pp. 195-223 (1995)). These approaches block the effects of TNF-α and IL-1 by either protein sequestration or receptor antagonism.

U.S. Pat. No. 5,100,897, incorporated herein by reference in its entirety, describes pyrimidinone compounds useful as angiotensin II antagonists wherein one of the pyrimidinone ring nitrogen atoms is substituted with a substituted phenylmethyl or phenethyl radical.

U.S. Pat. No. 5,162,325, incorporated herein by reference in its entirety, describes pyrimidinone compounds useful as angiotensin II antagonists wherein one of the pyrimidinone ring nitrogen atoms is substituted with a substituted phenylmethyl radical.

EP 481448, incorporated herein by reference in its entirety, describes pyrimidinone compounds useful as angiotensin II antagonists wherein one of the pyrimidinone ring nitrogen atoms is substituted with a substituted phenyl, phenylmethyl or phenethyl radical.

CA 2,020,370, incorporated herein by reference in its entirety, describes pyrimidinone compounds useful as angiotensin II antagonists wherein one of the pyrimidinone ring nitrogen atoms is substituted with a substituted biphenylaliphatic hydrocarbon radical.

### BRIEF DESCRIPTION OF THE INVENTION

The present invention comprises a new class of compounds useful in the prophylaxis and treatment of diseases, such as TNF-α, IL-1β, IL-6 and/or IL-8 mediated diseases and other maladies, such as pain and diabetes. In particular, the compounds of the invention are useful for the prophylaxis and treatment of diseases or conditions involving inflammation. Accordingly, the invention also comprises pharmaceutical compositions comprising the compounds; methods for the prophylaxis and treatment of TNF-α, IL-1β, IL-6 and/or IL-8 mediated diseases, such as inflammatory, pain and diabetes diseases, using the compounds and compositions of the invention, and intermediates and processes useful for the preparation of the compounds of the invention.

The compounds of the invention are represented by the following general structure: 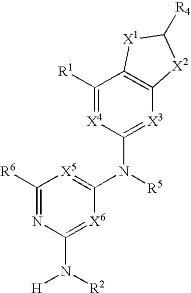
wherein R¹, R², R⁴, R⁵, R⁶, X¹, X², X³, X⁴, X⁵ and X⁶ are defined herein.

The foregoing merely summarizes certain aspects of the invention and is not intended, nor should it be construed, as limiting the invention in any way. All patents and other publications recited herein are hereby incorporated by reference in their entirety.

### DETAILED DESCRIPTION OF THE INVENTION

In accordance with the present invention, there is provided compounds of the formula: 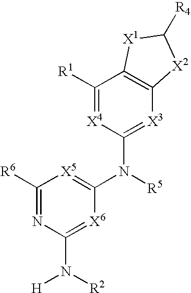
or a pharmaceutically acceptable salt or hydrate thereof, wherein

—X¹—CR⁴—X²— is selected from —N═CR⁴—NR^{a}—, —NR^{a}—CR⁴═N—, —N═CR⁴—O—, —O—CR⁴═N—, —N═CR⁴—S—, —S—CR⁴═N—, —N═CR⁴—CR⁴— and —CR⁴—CR⁴═N—;

X³ is N or CR⁴;

X⁴ is N or CR⁴;

X⁵ is N or CR⁵;

X⁶ is N or CR⁶; wherein only 1, 2 or 3 of X¹, X², X³ and X⁴ are N;

R¹ is a saturated, partially saturated or unsaturated 5-, 6- or 7-membered, ring containing 0, 1, 2 or 3 atoms selected from N, O and S, wherein the ring is substituted by 0, 1, 2 or 3 substituents selected from C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};

R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, N(R^{a})C(═O)OR^{b}, —N(R^{a})C(═O)NR^{a}R^{a}, N(R^{a})C(═NR^{a}) NR^{a}R^{a}, N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{e})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; or

R² is a saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, —OC(═O)R^{b}, —OC(═O)NR^{a}R^{b}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, N(R^{a})C(═NR^{a})—NR^{a}R^{a}, N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, NR^{a}R^{e}, N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I;

R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})—C(═O)R^{b}, —N(R^{a})C(═O)OR^{b}, —N(R^{a})C(═O)NR^{a}R^{a}, —N(R^{a})C(═NR^{a})NR^{a}R^{a}, N(R^{a})—S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};

R⁴ is independently in each instance H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, OR^{b}, OR^{e}OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, NR^{a}R^{a}, NR^{a}R^{e}, N(R^{a})C(═O)R^{b}, N(R^{a})C(═O)—OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)—₂NR^{a}R^{a}, —NR^{a}C₂₋₆alkyNR^{a}R^{a} or —NR^{a}C₂₋₆alkylOR^{a};

R⁵ is H, R^{e}, C₁₋₄haloalkyl, —C(═O)R^{b}, —C(═O)OR^{b}, —C(═O)NR^{a}R^{a} or C(═NR^{a})NR^{a}R^{a};

R⁶ is independently in each instance H, C₁₋₉alkyl, C₁₋₄haloalkyl, —NR^{a}R^{a}, —OR^{a}, or halo;

R^{a} is independently, at each instance, H or R^{b};

R^{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;

R^{d} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};

R^{e} is independently at each instance C₁₋₆alkyl substituted by 0, 1, 2 or 3 substituents independently selected from R^{d} and additionally substituted by 0 or 1 substituents selected from R^{g}; and

R^{g} is independently at each instance a saturated, partially saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, wherein the carbon atoms of the ring are substituted by 0, 1 or 2 oxo groups and the ring is substituted by 0, 1, 2 or 3 substituents selected from R^{b}, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

In another embodiment, in conjunction with the above and below embodiments, R¹ is a saturated or unsaturated 5- or 6-membered, ring containing 0, 1, 2 or 3 atoms selected from N, O and S, wherein the ring is substituted by 1, 2 or 3 substituents selected from C₁₋₄alkyl, C₁₋₄haloalkyl, halo, cyano, nitro, —C(═O)R^{b}, —C(═O)OR^{b}, C(═O)NR^{a}R^{a}, C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R¹ is a saturated or unsaturated 5- or 6-membered, ring containing 0, 1, 2 or 3 atoms selected from N, O and S, wherein the ring is substituted by 1, 2 or 3 substituents selected from C₁₋₄alkyl, C₁₋₄haloalkyl, halo, cyano, nitro, —OR^{a}, —OC(═O)R^{b}, —SR^{a}, —S(═O)R^{b}, —S(═O)₂R^{b}, —NR^{a}R^{a} and —N(R^{a})C(═O)R^{b}.

In another embodiment, in conjunction with the above and below embodiments, R¹ is a saturated or unsaturated 5- or 6-membered, ring containing 0, 1, 2 or 3 atoms selected from N, O and S, wherein the ring is substituted by 0, 1, 2 or 3 substituents selected from C₁₋₄alkyl, C₁₋₄haloalkyl and halo.

In another embodiment, in conjunction with the above and below embodiments, R¹ is a saturated or unsaturated 6-membered, ring containing 0, 1, 2 or 3 atoms selected from N, O and S, wherein the ring is substituted by 0, 1, 2 or 3 substituents selected from C₁₋₄alkyl, C₁₋₄haloalkyl and halo.

In another embodiment, in conjunction with the above and below embodiments, R¹ is phenyl substituted by 0, 1, 2 or 3 substituents selected from C₁₋₄alkyl, C₁₋₄haloalkyl and halo.

In another embodiment, in conjunction with the above and below embodiments, R¹ is phenyl.

In another embodiment, in conjunction with the above and below embodiments, R¹ is phenyl substituted by 1, 2 or 3 substituents selected from C₁₋₄alkyl, C₁₋₄haloalkyl and halo.

In another embodiment, in conjunction with the above and below embodiments, R¹ is pyridinyl substituted by 0, 1, 2 or 3 substituents selected from C₁₋₄alkyl, C₁₋₄haloalkyl and halo.

In another embodiment, in conjunction with the above and below embodiments, R¹ is pyrimidinyl substituted by 0, 1, 2 or 3 substituents selected from C₁₋₄alkyl, C₁₋₄haloalkyl and halo.

In another embodiment, in conjunction with the above and below embodiments, R¹ is a saturated or unsaturated 5-membered, ring containing 1 or 2 atoms selected from N, O and S, wherein the ring is substituted by 0, 1, 2 or 3 substituents selected from C₁₋₄alkyl, C₁₋₄haloalkyl and halo.

In another embodiment, in conjunction with the above and below embodiments, R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, N(R^{a})C(═O)OR^{b}, N(R^{a})C(═O)NR^{a}R^{a}, N(R^{a})C(═NR^{a}) NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C—(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{a}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is C₁₋₈alkyl.

In another embodiment, in conjunction with the above and below embodiments, R² is C₁₋₈alkyl substituted by 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a}) NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}R^{a}, C₂₋₆(═O)R^{g}, —C(═O)OR⁹, —C(═O)NR^{a}R^{g}, —C(═NR^{a})NR^{a}R^{g}, —OR⁹, —OC(═O)R^{g}, —OC(═O)NR^{a}R^{g}, —OC(═O)N(R^{g}, —S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, OC(═O)R^{b}, —OC(═O)NR^{a}R^{a}, —OC(═O)N(R^{a})S(═O)₂R^{b}, —OC₂₋₆alkylNR^{a}R, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, —N(R^{a})C(═O)R^{b}, N(R^{a})C(═O)OR^{b}, N(R^{a})C(═O)NR^{a}R^{a}, N(R^{a})C(═NR^{a}) NR^{a}R^{a}, N(R^{a})S(═O)₂R^{b}, —N(R^{a})S(═O)₂NR^{a}R^{a}, —NR^{a}C₂₋₆alkylNR^{a}R^{a}, —OC(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is C₂₋₈alkyl substituted by 0, 1, 2 or 3 substituents selected from C₁₋₂haloalkyl, halo, oxo, cyano, nitro, —C(═O)R^{b}, C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, N(R^{a})C(═O)R^{b}, N(R^{a})C(═O)OR^{b}, N(R^{a})C(═O)NR^{a}R^{a}, N(R^{a})C(═NR^{a}) NR^{a}R, —N(R^{a})S(═O)₂R^{b}, —N(R^{a})S(═O)₂NR^{a}R^{a}, —NR^{a}C₂₋₆alkylNR^{a}R^{g}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, C(═NR^{a})NR^{a}R^{a}, OR^{a}, OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is a saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, OC(═O)R^{b}, —OC(═O)NR^{a}R^{a}, —OC(═O)N(R^{a})S(═O)₂R^{b}, —OC₂₋₆alkylNR^{a}R, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, N(R^{a})C(═O)R^{b}, N(R^{a})C(═O)OR^{b}, —N(R^{a})C(═O)NR^{a}R^{a}, —N(R^{a})C(═NR^{a})—NR^{a}R^{a}, —N(R^{a})S(═O)₂R^{b}, —N(R^{a})S(═O)₂NR^{a}R, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR, —S(═O)R^{e}, —S(═O)₂R^{e}, S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated, partially saturated or unsaturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)—OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{g}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{e}, —NR^{a}R, —N(R^{a})C(═O)R^{g}, —N(R^{a})C(═O)OR^{g}, —N(R^{a})C(═O)NR^{a}R^{g}, —C(═O)R^{e}, C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}. —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})—C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O) R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)—OR^{b}, C(═O)NR^{a}R^{a}, C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, C(═O)OR^{e}, C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})—C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)—OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, C(═O)OR^{e}, C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})—C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 1 or 2 C₁₋₈alkyl groups, each being substituted by 1, 2 or 3 substituents selected from C₁₋₂haloalkyl, halo, oxo, cyano, nitro, —C(═O)R^{b}, —C(═O)—OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, C(═O)OR^{e}, C(═O)NR^{a}R^{a}, C(═NR^{a})NR^{a}R^{a}, —OR^{e}, —OC(═O)R^{e}, —OC(═O)NR^{a}R^{e}, —OC(═O)N(R^{a})S(═O)₂R^{e}, —OC₂₋₆alkylOR^{e}, —SR^{e}, S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})—C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1 or 2 N atoms, wherein the carbon atoms of the rings are substituted by 0, 1 or 2 oxo groups and the rings is substituted by 1, 2 or 3 substituents selected from R^{e}, R^{g}, C₁₋₈alkyl, C₁₋₄haloalkyl, cyano, nitro, —C(═O)R^{b}, C(═O)OR^{b}, —C(═O)NR^{a}R^{a}, C(═NR^{a})NR^{a}R^{a}, —ORB, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, N(R^{a})C(═O)R^{b}, N(R^{a})C(═O)OR^{b}, —N(R^{a})C(═O)NR^{a}R^{a}, N(R^{a})C(═NR^{a})—NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR, —N(R^{a})C(═O)NR^{a}R^{g}, —C(═O)R^{e}, —C(═O)OR^{e}, C(═O)NR^{a}R^{g}, —C(═NR^{a})NR^{a}R^{a}, OR^{e}, OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, NR^{a}R^{e}, N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})—NR^{a}R^{a}, OR^{b}, OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, —NR^{a}R^{e}, —N(R^{a})C(═O)R^{b}, N(R^{a})C(═O)OR^{b}, N(R^{a})C(═O)NR^{a}R^{a}, —N(R^{a})—C(═NR^{a})NR^{a}R^{a}, —N(R^{a})S(═O)₂R^{b}, —N(R^{a})S(═O)₂NR^{a}R^{a}, —NR^{a}C₂₋₆alkyl NR^{a}R^{a} and —NR^{a}C₂₋₆alkylOR^{a}.

In another embodiment, in conjunction with the above and below embodiments, R³ is H.

In another embodiment, in conjunction with the above and below embodiments, R³ is independently, in each instance, selected from H, C₁₋₆alkyl, C₁₋₄haloalkyl and halo.

In another embodiment, in conjunction with the above and below embodiments, R³ is independently, in each instance, selected from R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —NR^{a}R^{a}, —N(R^{a})—C(═O)R^{b}, —N(R^{a})C(═O)OR^{b}, —N(R^{a})C(═O)NR^{a}R^{a}, —N(R^{a})C(═NR^{a})NR^{a}R^{a}, N(R^{a})—S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with any of the above and below embodiments, R⁴ is independently in each instance R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})—C(═O)R^{b}, —N(R^{a})C(═O)OR^{b}, —N(R^{a})C(═O)NR^{a}R^{a}, —N(R^{a})C(═NR^{a})NR^{a}R^{a}, N(R^{a})—S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

In another embodiment, in conjunction with any of the above and below embodiments, R⁴ is H.

In another embodiment, in conjunction with any of the above and below embodiments, R⁵ is H.

In another embodiment, in conjunction with any of the above and below embodiments, R⁵ is R^{e}, C₁₋₄haloalkyl, —C(═O)R^{b}, —C(═O)OR^{b}, —C(═O)NR^{a}R^{a} or —C(═NR^{a})NR^{a}R^{a}.

In another embodiment, in conjunction with any of the above and below embodiments, R⁶ is H.

In another embodiment, in conjunction with any of the above and below embodiments, R⁶ is independently in each instance C₁₋₈alkyl, C₁₋₄haloalkyl, —NR^{a}R^{a}, —OR^{a}, or halo.

In another embodiment, in conjunction with any of the above and below embodiments, —X¹═X²— is —C(═O)—N(R^{a})— or —N(R^{a})—C(═O)—.

In another embodiment, in conjunction with any of the above and below embodiments, X¹ is N or CR³ and X² is N or CR⁴.

In another embodiment, in conjunction with any of the above and below embodiments, X¹ is CR³ and X² is N.

In another embodiment, in conjunction with any of the above and below embodiments, X¹ is N and X² is CR⁴.

In another embodiment, in conjunction with any of the above and below embodiments, X¹ is CR³ and X² is CR⁴.

In another embodiment, in conjunction with any of the above and below embodiments, X³ is N and X⁴ is CR⁴.

In another embodiment, in conjunction with any of the above and below embodiments, X³ is CR⁴ and X⁴ is N.

In another embodiment, in conjunction with any of the above and below embodiments, X³ is N and X⁴ is N.

In another embodiment, in conjunction with any of the above and below embodiments, X⁵ is N and X⁶ is CR⁶.

In another embodiment, in conjunction with any of the above and below embodiments, X⁵ is CR⁵ and X⁶ is N.

In another embodiment, in conjunction with any of the above and below embodiments, X⁵ is CR⁵ and X⁶ is CR⁶.

Another aspect of the invention relates to a pharmaceutical composition comprising a compound according to any one of the above embodiments and a pharmaceutically acceptable carrier.

Another aspect of the invention relates to a method of prophylaxis or treatment of inflammation comprising administering an effective amount of a compound according to any one of the above embodiments.

Another aspect of the invention relates to a method of prophylaxis or treatment of rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount of a compound according to any one of the above embodiments.

Another aspect of the invention relates to a method of lowering plasma concentrations of either or both TNF-α and IL-1 comprising administering an effective amount of a compound according to any one of the above embodiments.

Another aspect of the invention relates to a method of lowering plasma concentrations of either or both IL-6 and IL-8 comprising administering an effective amount of a compound according to any one of the above embodiments.

Another aspect of the invention relates to a method of prophylaxis or treatment of diabetes disease in a mammal comprising administering an effective amount of a compound according to any one of the above embodiments to produce a glucagon antagonist effect.

Another aspect of the invention relates to a method of prophylaxis or treatment of a pain disorder in a mammal comprising administering an effective amount of a compound according to any one of the above embodiments.

Another aspect of the invention relates to a method of decreasing prostaglandins production in a mammal comprising administering an effective amount of a compound according to any one of the above embodiments.

Another aspect of the invention relates to a method of decreasing cyclooxygenase enzyme activity in a mammal comprising administering an effective amount of a compound according to any one of the above embodiments. In another embodiment, the cyclooxygenase enzyme is COX-2.

Another aspect of the invention relates to a method of decreasing cyclooxygenase enzyme activity in a mammal comprising administering an effective amount of the above pharmaceutical composition. In another embodiment the cyclooxygenase enzyme is COX-2.

Another aspect of the invention relates to the manufacture of a medicament comprising a compound according to any one of the above embodiments.

Another aspect of the invention relates to the manufacture of a medicament for the treatment of inflammation comprising administering an effective amount of a compound according to any one of the above embodiments.

Another aspect of the invention relates to the manufacture of a medicament for the treatment of rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount of a compound according to any one of the above embodiments.

The compounds of this invention may have in general several asymmetric centers and are typically depicted in the form of racemic mixtures. This invention is intended to encompass racemic mixtures, partially racemic mixtures and separate enantiomers and diasteromers.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

Unless otherwise specified, the following definitions apply to terms found in the specification and claims:

“Aryl” means a phenyl or naphthyl radical, wherein the phenyl may be fused with a C₃₋₄cycloalkyl bridge.

“Benzo group”, alone or in combination, means the divalent radical C₄H₄═, one representation of which is —CH═CH—CH═CH—, that when vicinally attached to another ring forms a benzene-like ring—for example tetrahydronaphthylene, indole and the like.

“C_{α-β}alkyl” means an alkyl group comprising from α to β carbon atoms in a branched, cyclical or linear relationship or any combination of the three. The alkyl groups described in this section may also contain double or triple bonds. Examples of C₁₋₈alkyl include, but are not limited to the following: 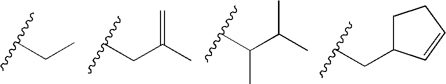
“Halogen” and “halo” mean a halogen atoms selected from F, Cl, Br and I. “C_{α-β}haloalkyl” means an alkyl group, as described above, wherein any number—at least one—of the hydrogen atoms attached to the alkyl chain are replaced by F, Cl, Br or I.
“Heterocycle” means a ring comprising at least one carbon atom and at least one other atom selected from N, O and S. Examples of heterocycles that may be found in the claims include, but are not limited to, the following: 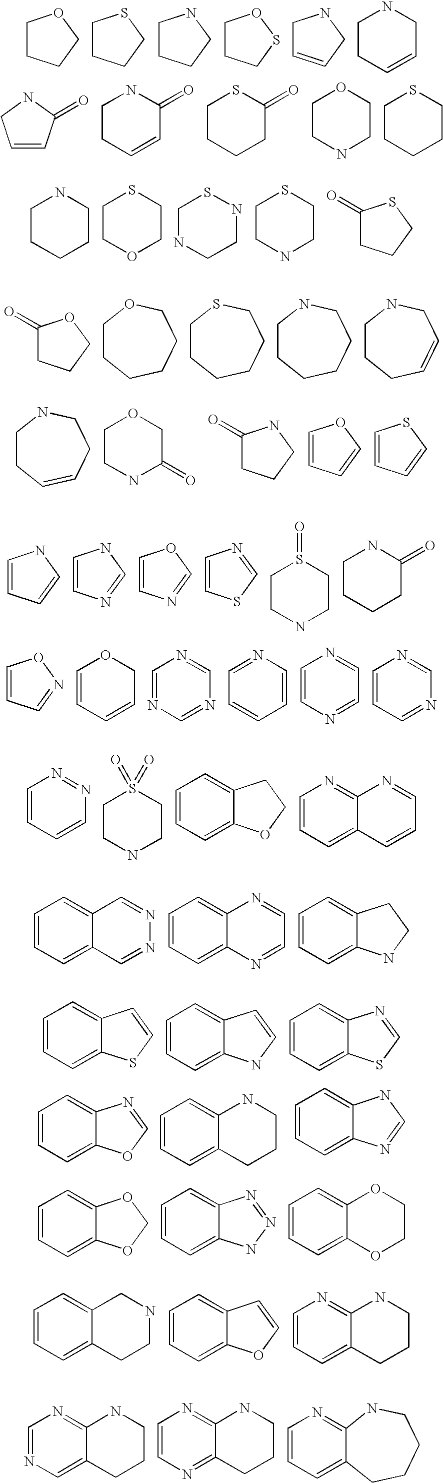 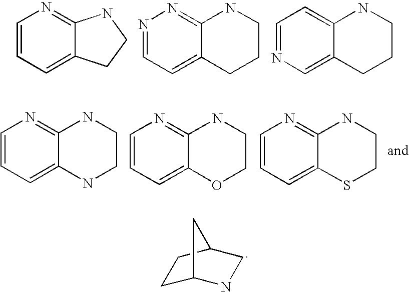
“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulphonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).
“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.
“Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, tri-fluoroacetyl, tri-chloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, tri-isopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-tri-silyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydroylsis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′=O, S, NR), and the like, which are illustrated in the following examples: 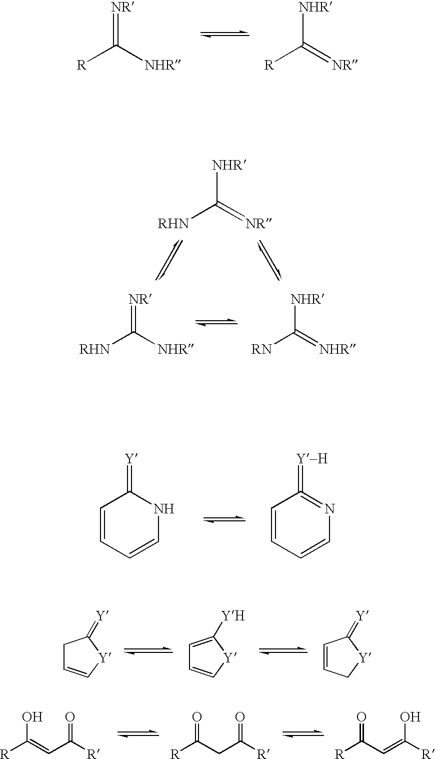
and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

“Cytokine” means a secreted protein that affects the functions of other cells, particularly as it relates to the modulation of interactions between cells of the immune system or cells involved in the inflammatory response. Examples of cytokines include but are not limited to interleukin 1 (IL-1), preferably IL-1β, interleukin 6 (IL-6), interleukin 8 (IL-8) and TNF, preferably TNF-α (tumor necrosis factor-α).

“TNF, IL-1, IL-6, and/or IL-8 mediated disease or disease state” means all disease states wherein TNF, IL-1, IL-6, and/or IL-8 plays a role, either directly as TNF, IL-1, IL-6, and/or IL-8 itself, or by TNF, IL-1, IL-6, and/or IL-8 inducing another cytokine to be released. For example, a disease state in which IL-1 plays a major role, but in which the production of or action of IL-1 is a result of TNF, would be considered mediated by TNF.

Compounds according to the invention can be synthesized according to one or more of the following methods. It should be noted that the general procedures are shown as it relates to preparation of compounds having unspecified stereochemistry. However, such procedures are generally applicable to those compounds of a specific stereochemistry, e.g., where the stereochemistry about a group is (S) or (R). In addition, the compounds having one stereochemistry (e.g., (R)) can often be utilized to produce those having opposite stereochemistry (i.e., (S)) using well-known methods, for example, by inversion.

Abbreviations

Ac₂O acetic anhydride
CH₂Cl₂ dichloromethane, methylene chloride
DCM dichloromethane
DCE 1,2-dichloroethane
DME dimethoxyethane, ethylene glycol dimethyl ether
DMF dimethyl formamide
EDC 1-[3-(dimethylamino)propyl]-3-ethylcarbodiimide hydrochloride
Et₂O diethyl ether
EtOH ethanol
EtOAc ethyl acetate
Fmoc 9-fluorenylmethoxycarbonyl
h hour(s)
MeOH methanol
NMP 1-methyl-2-pyrrolidinone
i-PrOH isopropanol
PS-carbodiimide polymer supported carbodiimide resin from Argonaut
RT room temperature
SiO₂ silica
TFA trifluoroacetic acid
THF tetrahydrofuran

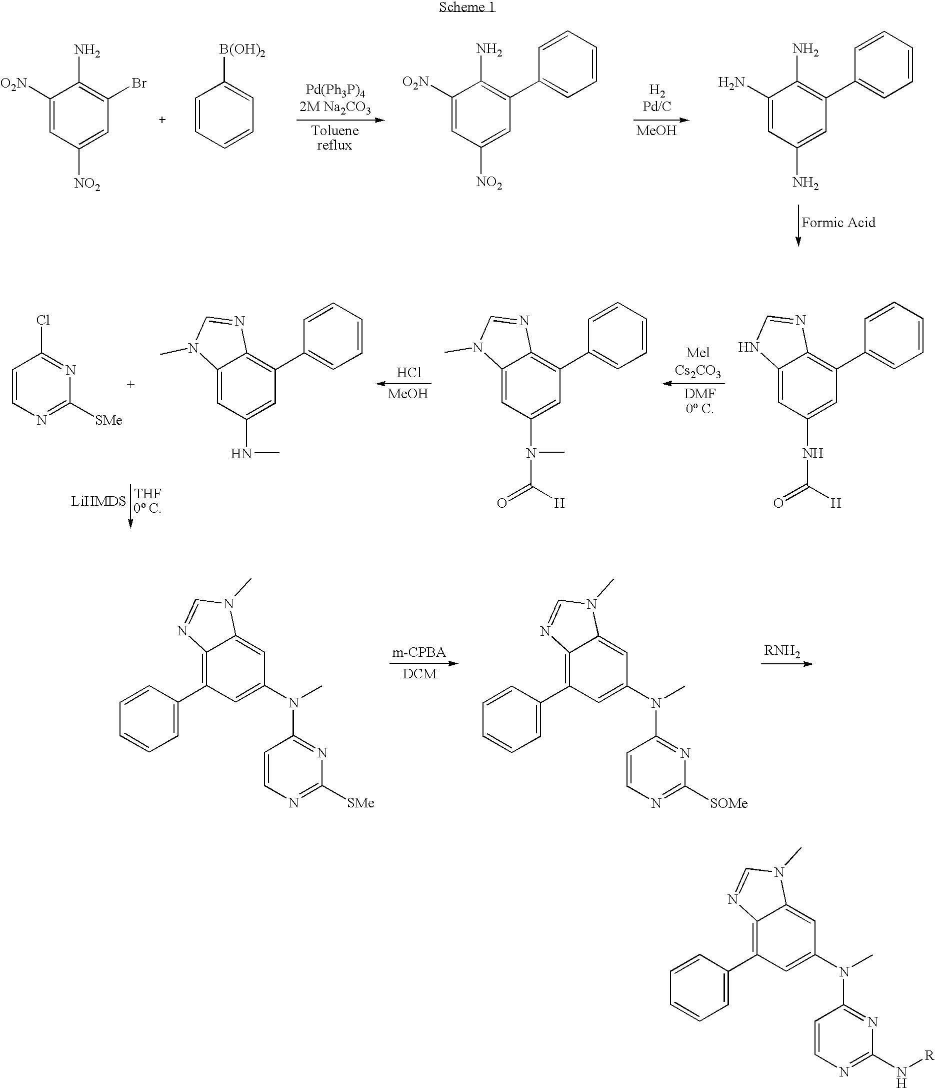

### EXAMPLE 1

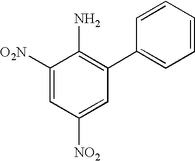 2-Phenyl-4,6-dinitrobenzenamine: To a 500 mL flask was charged 2-bromo-4,6-dinitroaniline (5.0 g, 19.1 mmol), phenylboronic acid (3.5 g, 28.6 mmol), tetrakis(triphylphosphine) palladium(0) (1.1 g, 0.96 mmol), 2M sodium carbonate (20 mL) and toluene (150 mL). Reaction heated to reflux for 21 h. The reaction was diluted with ethyl acetate (150 mL) and 5% NaHCO₃ (50 mL). The organic layer was further washed with saturated NH₄Cl, then washed through silica (150 g) washing with ethyl acetate (100 mL). The organic layer was concentrated to dryness under reduced pressure, and crystallization induced with methanol to afford yellow crystals. M−1=258.

### EXAMPLE 2

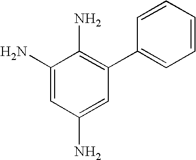 2-Phenyl-4,6-diaminobenzenamine: To a stirring suspension of 2-phenyl-4,6-dinitrobenzenamine (4.8 g, 18.5 mmol) in methanol (150 mL) was added 5% palladium on carbon (200 mg). The reaction was stirred overnight under an atmosphere of hydrogen at 25° C. The reaction was filtered through a bed of Celite then concentrated to a solid under reduced pressure. M+1=200.

### EXAMPLE 3

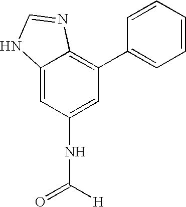 N-(7-Phenyl-3H-benzo[d]imidazol-5-yl)formamide: A 100 mL flask was charged with 2-phenyl-4,6-diaminobenzenamine (3.4 g, 17.1 mmol) and formic acid (30 mL) then heated to reflux for 1 h. The solvent was removed under reduced pressure. The resulting residue was purified on silica eluting with 2M ammonia in methanol/dichloromethane. The product was isolated as a red solid. M+1=238. A side product of 6-nitro-4-phenyl-1H-benzo[d]imidizole was obtained from this reaction. M+1=240.

### EXAMPLE 4

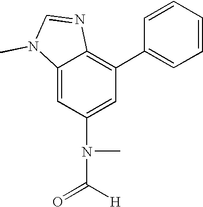 N-Methyl-N-(3-methyl-7-phenyl-3H-benzo[d]imidazol-5-yl)formamide and N-methyl-N-(1-methyl-7-phenyl-1H-benzo[d]imidazol-5-yl)formamide: To a stirring solution of N-(7-phenyl-3H-benzo[d]imidazol-5-yl)formamide (0.65 g, 2.7 mmol) in N,N-dimethylformamide (25 mL) was added cesium carbonate (2.7 g, 8.2 mmol). The reaction was cooled to 0° C. and methyl iodide (0.52 mL, 8.2 mmol) was added. The ice bath was removed and the reaction was stirred for 3 h. The solvent was removed under reduced pressure and the residue was partitioned between dichloromethane (50 mL) and 1N sodium hydroxide (20 mL). The organic layer was then washed with brine and dried over magnesium sulfate. The product was purified on silica eluting with 2-3% methanol/dichloromethane. Regioisomers were separated on silica. N-Methyl-N-(3-methyl-7-phenyl-3H-benzo[d]imidazol-5-yl)formamide isolated as dark orange foam. M+1=266.

### EXAMPLE 5

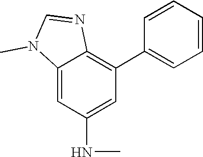 N,3-Dimethyl-7-phenyl-3H-benzo[d]imidazol-5-amine: To a stirring solution of N-methyl-N-(3-methyl-7-phenyl-3H-benzo[d]imidazol-5-yl)formamide (0.55 g, 2.1 mmol) in methanol (30 mL) was added 2M hydrochloric acid in diethylether (10 mL). The reaction was stirred for 4 h at 25° C. The solvent was removed under reduced pressure and the residue was partitioned between 1N sodium hydroxide (10 mL) and dichloromethane (30 mL). The organic layer was then washed with brine and dried over magnesium sulfate. The product was isolated as a dark solid. M+1=238.

### EXAMPLE 6

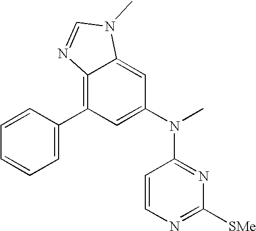 N,3-Dimethyl-N-(2-(methylthio)pyrimidin-4-yl)-7-phenyl-3H-benzo[d]imidazol-5-amine: To a stirring solution of N,3-dimethyl-7-phenyl-3H-benzo[d]imidazol-5-amine (1.07 g, 4.5 mmol) in tetrahydrofuran (10 mL) at 0° C. under nitrogen was added lithium bis(trimethylsilyl)amide (1 M in THF; 5.4 mL). After 2 min, 4-chloro-2-(methylthio)pyrimidine (0.58 mL, 5.0 mmol) was added to the reaction. The solvent was removed under reduced pressure, and the residue carried on without further purification. M+1=362.

### EXAMPLE 7

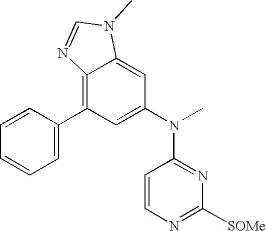 N,3-Dimethyl-N-(2-(methylsulfinyl)pyrimidin-4-yl)-7-phenyl-3H-benzo[d]imidazol-5-amine: To a solution of crude N,3-dimethyl-N-(2-(methylthio)pyrimidin-4-yl)-7-phenyl-3H-benzo[d]imidazol-5-amine (4.5 mmol) in dichloromethane (30 mL) was added 3-chloroperoxybenzoic acid (1.8 g, 11 mmol) at 25° C. The reaction was stirred for 30 min then diluted with dichloromethane (70 mL) and isopropyl alcohol (5 mL) and washed with 5% sodium carbonate (50 mL). The aqueous layer was washed once with dichloromethane (20 mL) and the combined organics were further washed with brine then dried over magnesium sulfate. The product was then purified on silica, and isolated as amber foam. M+1=362.

### EXAMPLE 8

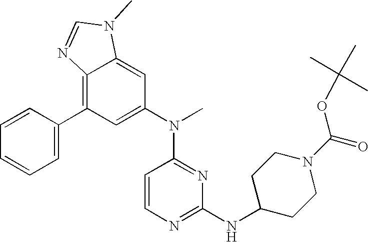 tert-Butyl 4-(4-(methyl(3-methyl-7-phenyl-3H-benzo[d]imidazol-5-yl)amino)-pyrimidin-2-ylamino)piperidine-1-carboxylate: To a 4 mL vessel was charged N,3-dimethyl-N-(2-(methylsulfinyl)pyrimidin-4-yl)-7-phenyl-3H-benzo[d]imidazol-5-amine (120 mg, 0.33 mmol), tert-butyl 4-aminopiperidine-1-carboxylate (133 mg, 0.66 mmol), and 1,4-dioxane (0.75 mL). The vessel was capped and heated to 95° C. for 18 h. The solvent was removed under reduced pressure and the residue was purified on silica. The product was isolated as colorless film. M+1=514. ¹H NMR (CDCl₃) s (9H, 1.45 ppm), d (2H, 2.06 ppm), m (2H, 2.94 ppm), s (3H, 3.51 ppm), s (3H, 3.87 ppm), m (4H, 4.00 ppm), d (2H, 4.80 ppm), d (1H, 5.67 ppm), s (1H, 7.24 ppm), s (1H, 7.32 ppm), m (1H, 7.40 ppm), t (2H, 7.50 ppm), d (1H, 7.72 ppm), m (3H, 7.98 ppm).

### EXAMPLE 9

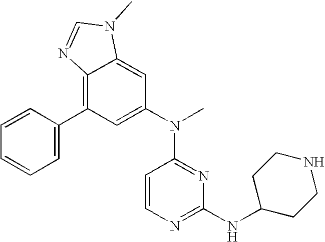 N4-Methyl-N-4-(3-methyl-7-phenyl-3H-benzo [d] imidazol-5-yl)-N-2-(piperidin-4-ylmethyl)pyrimidine-2,4-diamine: To a stirred solution of tert-butyl 4-(4-(methyl(3-methyl-7-phenyl-3H-benzo[d]imidazol-5-yl)amino)pyrimidin-2-ylamino)piperidine-1-carboxylate (60 mg, 0.12 mmol) in dichloromethane (1 mL) was added trifluoro-acetic acid (1 mL) at 25° C. and the reaction was stirred for 3 h. The solvent was removed under reduced pressure and residue was partitioned between dichloro-methane (10 mL) and 1N sodium hydroxide (5 mL). The organic layer was dried with magnesium sulfate then purified on silica. The product was lyophilized from 50% aqueous acetonitrile (2 mL) to obtain a fluffy white powder. M+1=414 ¹H NMR (CDCl₃) dd (2H, 1.44 ppm), d (2H, 2.11 ppm), s (2H, 2.59 ppm), t (2H, 2.75 ppm), d (2H, 3.13 ppm), s (1H, 3.48 ppm), s (3H, 3.53 ppm), s (3H, 3.87 ppm), d (1H, 3.95 ppm), d (1H, 5.66 ppm), s (1H, 7.24 ppm), s (1H, 7.26 ppm), m (1H, 7.32 ppm), m (2H, 7.39 ppm), d (1H, 7.70 ppm), m (3H, 7.98 ppm).

### EXAMPLE 10

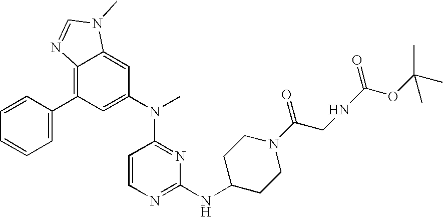 tert-Butyl 2-(4-((4-(methyl(3-methyl-7-phenyl-3H-benzo[d]imidazol-5-yl)amino)-pyrimidin-2-ylamino)methyl)piperidin-1-yl)-2-oxoethylcarbamate: To a stirring solution of N4-methyl-N-4-(3-methyl-7-phenyl-3H-benzo[d]imidazol-5-yl)-N-2-(piperidin-4-ylmethyl)pyrimidine-2,4-diamine (30 mg, 0.073 mmol) in dichloromethane (1 mL) was added N-(tert-butoxycarbonyl)glycine-N-hydroxysuccinimide ester (39 mg, 0.14 mmol) and the reaction was stirred for 10 min at 25° C. The product was purified on silica yielding a white solid. M+1=571.

### EXAMPLE 11

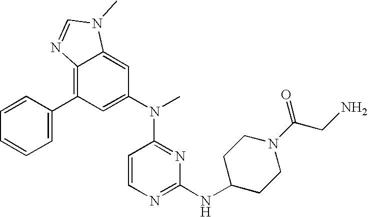 2-Amino-1-(4-((4-(methyl(3-methyl-7-phenyl-3H-benzo[d]imidazol-5-yl)amino)-pyrimidin-2-ylamino)methyl)piperidin-1-yl)ethanone: The compound was synthesized similar to that of N4-methyl-N-4-(3-methyl-7-phenyl-3H-benzo[d]imidazol-5-yl)-N-2-(piperidin-4-ylmethyl)pyrimidine-2,4-diamine. M+1=471.

The following molecules were synthesized similar to that of tert-Butyl 4-(4-(methyl(3-methyl-7-phenyl-3H-benzo [d] imidazol-5-yl)amino)pyrimidin-2-ylamino)piperidine-1-carboxylate:

### EXAMPLE 12

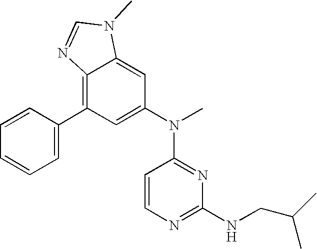 N2-isobutyl-N-4-methyl-N-4-(3-methyl-7-phenyl-3H-benzo[d]imidazol-5-yl)-pyrimidine-2,4-diamine: M+1=387 ¹H NMR (CDCl₃) s (3H, 0.98 ppm), s (3H, 0.99 ppm), m (1H, 1.92 ppm), t (2H, 3.26 ppm), s (3H, 3.53 ppm), s (3H, 3.86 ppm), s (1H, 4.97 ppm), d (1H, 5.64 ppm), m (1H, 7.24 ppm), s (1H, 7.25 ppm), m (1H, 7.36 ppm), m (2H, 7.50 ppm), d (1H, 7.71 ppm), m (3H, 7.98 ppm).

### EXAMPLE 13

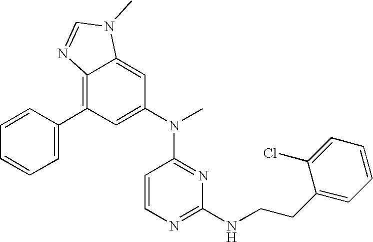 N2-(2-chlorophenethyl)-N-4-methyl-N-4-(3-methyl-7-phenyl-3H-benzo[d]imidazol-5-yl)pyrimidine-2,4-diamine: M+1=469. ¹H NMR (CDCl₃) m (2H, 2.87 ppm), m (2H, 2.96 ppm), m (2H, 3.09 ppm), s (3H, 3.55 ppm), m (2H, 3.70 ppm), s (3H, 3.86 ppm), s (1H, 5.02 ppm), d (1H, 5.67 ppm), m (3H, 7.17 ppm), m (3H, 7.25 ppm), m (3H, 7.34 ppm), m (2H, 7.49 ppm), d (1H, 7.71 ppm), m (3H, 7.99 ppm).

### EXAMPLE 14

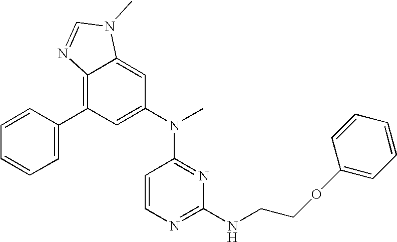 N-4-methyl-N-4-(3-methyl-7-phenyl-3H-benzo[d]imidazol-5-yl)-N-2-(2-phenoxy-ethyl)pyrimidine-2,4-diamine: M+1=451 ¹H NMR (CDCl₃) t (2H, 3.08 ppm), s (3H, 3.53 ppm), m (2H, 3.86 ppm), s (3H, 3.87 ppm), t (2H, 3.99 ppm), t (2H, 4.16 ppm), d (1H, 5.68 ppm), m (5H, 6.94 ppm), m (6H, 7.26 ppm), m (1H, 7.33 ppm), d (1H, 7.71 ppm), m (3H, 7.98 ppm).

### EXAMPLE 15

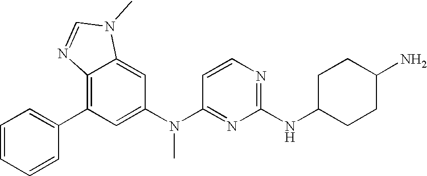 N2-((1 s,4s)-4-aminocyclohexyl)-N-4-methyl-N-4-(3-methyl-7-phenyl-3H-benzo[d]imidazol-5-yl)pyrimidine-2,4-diamine: M+1=428 ¹H NMR (CDCl₃) m (11H, 1.20 ppm), d (2H, 1.83 ppm), m (2H, 1.90 ppm), m (2H, 2.17 ppm), m (2H, 2.63-2.71 ppm), s (3H, 3.52 ppm), s (1H, 3.80 ppm), s (3H, 3.87 ppm), d (1H, 4.69 ppm), d (1H, 5.64 ppm), s (1H, 7.24 ppm), s (1H, 7.33 ppm) t (1H, 7.38 ppm), t (2H, 7.50 ppm), d (1H, 7.72 ppm), m (3H, 7.98 ppm).

### EXAMPLE 16

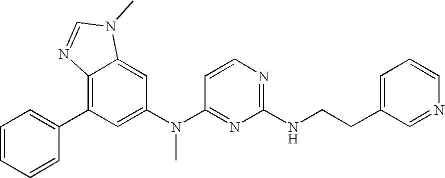 N4-methyl-N-4-(3-methyl-7-phenyl-3H-benzo[d]imidazol-5-yl)-N-2-(2-(pyridin-3-yl)ethyl)pyrimidine-2,4-diamine: M+1=436 ¹H NMR (CDCl₃) t (2H, 2.96 ppm), s (3H, 3.54 ppm), dd (2H, 3.69 ppm), s (3H, 3.87 ppm), s (1H, 5.08 ppm), d (1H, 5.69 ppm), m (2H, 7.25 ppm), s (1H, 7.33 ppm), t (1H, 7.39 ppm), m (3H, 7.50 ppm), d (1H, 7.71 ppm), s (2H, 7.97 ppm) d (1H, 7.99 ppm), d (1H, 8.47 ppm), s (1H, 8.53 ppm).

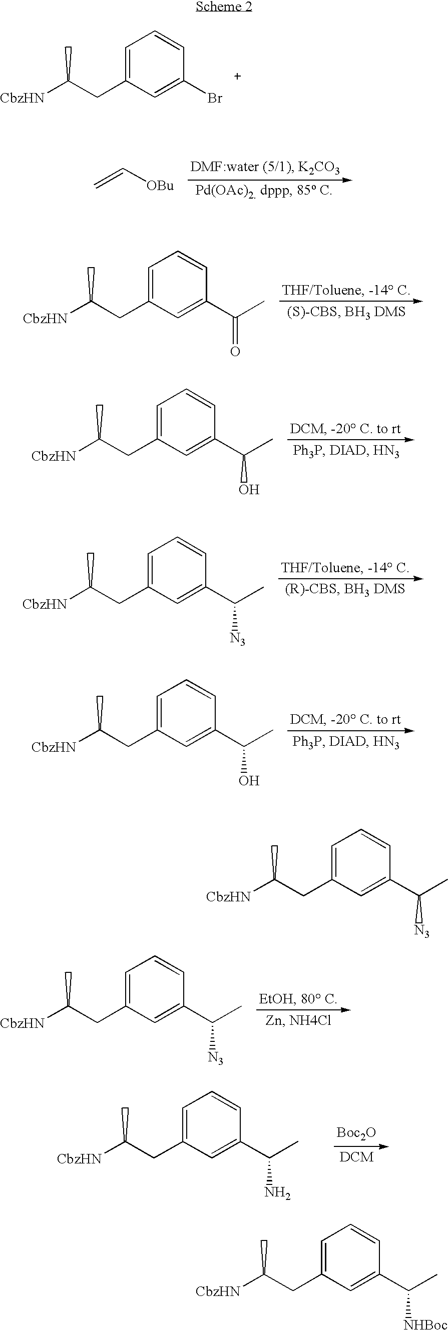

### EXAMPLE 17

(S)-Benzyl 1-(3-acetylphenyl)propan-2-ylcarbamate: To a round-bottom flask equipped with mechanical stirrer, were sequentially added (S)-benzyl 1-(3-bromo-phenyl)propan-2-ylcarbamate (81.97 g, 235 mmol), Pd(OAc)₂ (0.53, 2.35 mmol), dppp (2.13 g, 5.17 mmol), and K₂CO₃ (39.2 g, 284 mmol). The mixture was degassed (three times) by vacuum/N₂ backfills. To the mixture were added the pre-mixed solution of 5:1 DMF/water (0.7 L) and butyl vinyl ether (59.5 g, 590 mmol). The resulting mixture was heated to 85° C. for 30 min. The reaction mixture became clear with a second layer at the bottom containing black precipitate. The mixture was then stirred at 90° C. for 19 h. HPLC shows complete conversion. The reaction mixture was cooled to rt, CuCl (0.15 g, 1.5 mmol) was added. The mixture was placed in an ice-bath and 10% HCl (50 mL, 3.2 M) was slowly added. The mixture was stirred until all of solids were dissolved and bubbling ceased. To the mixture was added K₂CO₃ (39 g) in portions and left overnight without stirring. The resulting mixture was filtered and washed with DCM. The filtrate was concentrated under vacuum and the residue was dissolved in EtOAc (1 L) and then treated with active carbon (5 g), which was filtered over Celite. The filtrate (1.3 L) was washed with water (0.4 L) and brine. The combined aqueous layer was extracted with EtOAc (0.1 L). The combined brined washed organic layer was dried over MgSO₄ and filtered over Celite. The filtrate was concentrated under vacuum and the residue was dissolved in EtOAc (0.2 L) and treated with hexane until a dark-brown oil appeared on the wall of the flask. The oil was decanted and passed through a Celite pad, and then the filtrate (a yellow solution) was placed in a freezer. The product (yellowish tiny needles) was collected by filtration. The Celite pad containing the oil was rinsed with DCM and DCM was removed under vacuum. The residue was purified by silica gel chromatography (elution of hexanes/EtOAc from 3:1 to 1:1) to give product as yellowish tiny needles. In addition, more product was obtained after purification of the recovered material from the mother liquors by silica gel chromatography.

### EXAMPLE 18

Benzyl (S)-1-(3-((R)-1-hydroxyethyl)phenyl)propan-2-ylcarbamate: A flask (1 L, 3-necks) equipped with magnetic stir, addition funnel and temperature probe was flushed with N₂. To the flask were added dry toluene (100 mL), (S)-CBS reagent (1M in toluene, 5.9 mL, 5.9 mmol) and borane dimethylsulfide complex (neat, ca. 10M, 10.5 mL, 105 mmol). The mixture was cooled to −18° C. with an ice/acetone bath. To the cooled solution was added drop wise over 1.5 h to a solution of compound (S)-benzyl 1-(3-acetylphenyl)propan-2-ylcarbamate (31.1 g, 100 mmol) in THF (100 mL) while maintaining an internal temperature below −14° C. The addition funnel was rinsed with THF (15 mL), and the reaction mixture was allowed to warm to 0° C. and cool to −15° C. Methanol (40 mL) was carefully added to the resulting mixture (temperature rose to −5° C. and bubbling occurred). The mixture was warmed to room temperature and concentrated under vacuum. The residue was then dissolved in 1:1 EtOAc/Et₂O (40 mL), which was washed with 1M HCl (2×100 mL), water (100 mL), brine and dried over MgSO₄. The solvent was concentrated under vacuum to give product as a yellow wax.

### EXAMPLE 19

Benzyl (S)-1-3-((S)-1-azidoethyl)phenyl)propan-2-ylcarbamate: To a flask (1 L, 3-necks) equipped with magnetic stir, addition funnel and temperature probe was added triphenylphosphine (28.8 g, 110 mmol), dry DCM (100 mL) and the mixture was cooled to −30° C. To the cooled mixture was added drop wise DIAD (22.2 g, 110 mmol), a solution of HN₃ in toluene (150 mL, 1.1 M) and a solution of benzyl (S)-1-3-(R)-1-hydroxyethyl)phenyl)propan-2-ylcarbamate (100 mmol) in DCM (100 mL) while maintaining an internal temperature below −20° C. The reaction mixture was then warmed to 0° C. and stirred for 30 min. TLC showed the starting material benzyl (S)-1-3-((S)-1-hydroxyethyl)phenyl)propan-2-ylcarbamate was consumed. The reaction mixture was warmed to rt (bubbling occurred). The surface of the reaction mixture was blown with N₂. Methanol was added to the reaction mixture and the solvent was concentrated under vacuum to give product as a mixture of oil and crystals.

### EXAMPLE 20

Benzyl (S)-1-(3-((S)-1-aminoethyl)phenyl)propan-2-ylcarbamate: A mixture of Example 19 (88 g, 100 mmol), zinc (13 g, 0.2 mmol), ammonium chloride (21 g, 0.4 mol) in abs. ethanol (350 mL) was heated to 80° C. for 1.5 h. LC/MS showed complete conversion of the starting material. The solvent was removed under vacuum. The oily residue was extracted using 0.5M H₂SO₄ (4×0.20 L)—with each consecutive extraction the insoluble residue became more crystalline. The solid residue (mostly triphenylphosphine oxide and reduced DIAD) was discarded. The combined cloudy aqueous layers were treated with concentrated aq. NH₃ (150 mL) and extracted with DCM (3×0.20 L). The DCM solution was concentrated to a final volume of ca. 0.5 L, which was directly used to next step.

### EXAMPLE 21

Benzyl (S)-1-(3-((S)-1-N-Boc-aminoethyl)phenyl)propan-2-ylcarbamate: To a solution of benzyl (S)-1-(3-((S)-1-aminoethyl)phenyl)propan-2-ylcarbamate in DCM (Example 20) was added Boc₂O (17.5 g, 80 mmol) at room temperature. The mixture was stirred at room temperature overnight. The solvent was removed under reduced pressure to afford the product as an oily residue.

### EXAMPLE 22

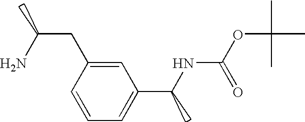 tert-Butyl (S)-1-(3-((S)-2-aminopropyl)phenyl)ethylcarbamate: To a stirring solution of benzyl (S)-1-(3-((S)-1-N-Boc-aminoethyl)phenyl)propan-2-ylcarbamate (4.0 g, 9.7 mmol) in methanol (40 mL) under nitrogen was added Pd/C (10%, 200 mg). The mixture was stirred for 1 h at 25° C. under an atmosphere of hydrogen. The reaction was filtered though Celite and reduced to a colorless oil under reduced pressure. M+1=279.

### EXAMPLE 23

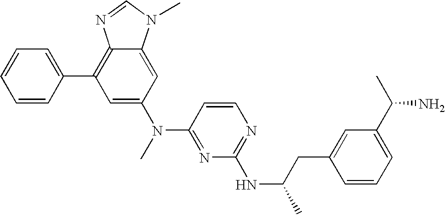 N2-((S)-1-(3-((S)-1-Aminoethyl)phenyl)propan-2-yl)-N-4-methyl-N-4-(3-methyl-7-phenyl-3H-benzo[d]imidazol-5-yl)pyrimidine-2,4-diamine (molecule made similar to that of tert-Butyl 4-(4-(methyl(3-methyl-7-phenyl-3H-benzo[d]imidazol-5-yl)-amino)pyrimidin-2-ylamino)piperidine-1-carboxylate and N4-Methyl-N-4-(3-methyl-7-phenyl-3H-benzo[d]imidazol-5-yl)-N-2-(piperidin-4-ylmethyl)pyrimidine-2,4-diamine) product isolated as a sulfuric acid salt. M+1=492.3. ¹H NMR (DMSO-d₆) d (3H, 1.26 ppm), d (3H, 1.48 ppm), s (9H, 2.35 ppm), m (1H, 2.85 ppm), m (1H, 3.01 ppm), s (3H, 3.63 ppm), s (3H, 3.92 ppm), m (2H, 4.95 ppm), d (1H, 5.77 ppm), m (5H, 7.33-7.39 ppm), m (3H, 7.44 ppm), s (1H, 7.65 ppm), s (1H, 7.78 ppm), d (2H, 8.06 ppm), s (3H, 8.06 ppm), m (1H, 8.23 ppm), s (1H, 8.62 ppm), s (1H, 11.80 ppm).

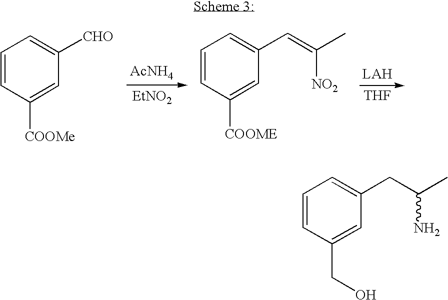

### EXAMPLE 24

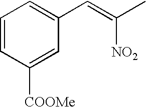 (Z)-Methyl 3-(2-nitroprop-1-enyl)benzoate: A 500 mL round bottom flask was charged methyl 3-formylbenzoate (2.60 g, 15.9 mmol), ammonium acetate (1.22 g, 15.9 mmol), and nitroethane (80 mL). The flask was heated to reflux for 1 h under nitrogen to create a solution. The solvent was removed under reduced pressure and the residue was partitioned between ethyl acetate (100 mL) and 5% sodium hydrogen carbonate (50 mL). The organic layer was washed with brine then dried over magnesium sulfate. The product was isolated as yellow crystals. ¹H NMR (CDCl₃) s (3H, 2.47 ppm), s (3H, 3.96 ppm), m (2H, 7.55-7.60 ppm), m (3H, 8.10 ppm).

### EXAMPLE 25

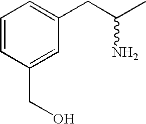 (3-(2-Aminopropyl)phenyl)methanol: To a cloudy suspension of 1M lithium aluminum hydride in tetrahydrofuran (THF) (45 mL, 45 mmol) at 0° C. under nitrogen was added a solution of (Z)-methyl 3-(2-nitroprop-1-enyl)benzoate (1.0 g, 4.5 mmol) in THF (5 mL). A slurry resulted and another 15 mL THF was added. The reaction was stirred overnight warming to room temperature. The reaction was cooled to 0° C., and a saturated solution of potassium sodium tartrate tetrahydrate was added (30 mL). The resulting solid was isolated by filtration and the solid was washed with THF (100 mL). The solvent was removed under reduced pressure and the residue was partitioned between dichloromethane (30 mL) and 1N sodium hydroxide (10 mL). The organic layer was purified on silica and isolated as a viscous colorless oil. M+1=166. ¹H NMR (CDCl₃) d (3H, 1.11 ppm), s (2H, 1.86 ppm), dd (1H, 2.50 ppm), dd (1H, 2.71 ppm), m (1H, 3.13 ppm), s (2H, 4.65 ppm), d (1H, 7.09 ppm), m (2H, 7.17 ppm), m (1H, 7.28 ppm).

### EXAMPLE 26

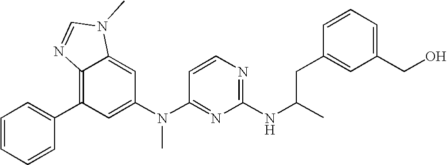 (3-(2-(4-(Methyl(3-methyl-7-phenyl-3H-benzo[d]imidazol-5-yl)amino)pyrimidin-2-ylamino)propyl)phenyl)methanol: The compound was synthesized similar to that of N4-methyl-N-4-(3-methyl-7-phenyl-3H-benzo[d]imidazol-5-yl)-N-2-(piperidin-4-ylmethyl)pyrimidine-2,4-diamine M+1=479 ¹H NMR (CDCl₃) d (3H, 1.19 ppm), dd (1H, 2.75 ppm), dd (1H, 3.03 ppm), s (3H, 3.48 ppm), d (1H, 3.51 ppm), s (2H, 3.56 ppm), s (2H, 3.85 ppm), d (1H, 3.87 ppm), d (1H, 4.62 ppm), d (1H, 5.66 ppm), d (1H, 7.13 ppm), m (2H, 7.20 ppm), d (1H, 7.25 ppm), m (2H, 7.32 ppm), m (1H, 7.39 ppm), m (2H, 7.50 ppm), d (1H, 7.63 ppm), m (3H, 7.97 ppm)

### EXAMPLE 27

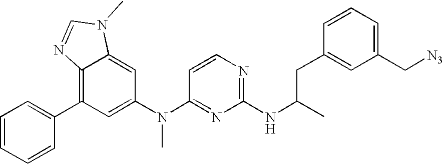 N2-(1-(3-(Azidomethyl)phenyl)propan-2-yl)-N-4-methyl-N-4-(3-methyl-7-phenyl-3H-benzo[d]imidazol-5-yl)pyrimidine-2,4-diamine: A 25 mL round bottom flask was charged with (3-(2-(4-(methyl(3-methyl-7-phenyl-3H-benzo[d]imidazol-5-yl)-amino)pyrimidin-2-ylamino)propyl)phenyl)methanol (260 mg, 0.54 mmol), diphenyl phosphoryl azide (0.23 mL, 1.1 mmol), 1,8-diazabicyclo[5.4.0]undec-7-ene (0.16 mL, 1.1 mmol), and THF (3 mL). The reaction mixture was stirred for 3 days under nitrogen. The solvent was removed under reduced vacuum, and the resulting residue was purified on silica eluting with 2.5% methanol/dichloromethane. M+1=504.

### EXAMPLE 28

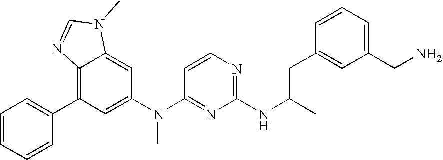 N2-(1-(3-(Aminomethyl)phenyl)propan-2-yl)-N-4-methyl-N-4-(3-methyl-7-phenyl-3H-benzo[d]imidazol-5-yl)pyrimidine-2,4-diamine: To a stirring solution of N2-(1-(3-(azidomethyl)phenyl)propan-2-yl)-N-4-methyl-N-4-(3-methyl-7-phenyl-3H-benzo[d]imidazol-5-yl)pyrimidine-2,4-diamine (295 mg, 0.59 mmol) in ethanol (6 mL) was added zinc (190 mg, 3.0 mmol), and a saturated solution of ammonium chloride (0.3 mL). The mixture was heated to reflux for 3 h. The sovent was removed under reduced pressure and the resulting residue was partitioned between DCM (30 mL) and 1 N NaOH (10 mL). The aqueous layer was washed three times with DCM (10 mL). The combined organic extracts were dried with brine and magnesium sulfate, then purified on silica. The product was further purified by reverse phase chromatography (C-18) eluting with water and acetonitrile (0.1% TFA). The isolated product then was partitioned between DCM (50 mL) and 1N NaOH (5 mL). The aqueous layer was washed three times with DCM (5 mL). The combined organic extracts were dried with brine and magnesium sulfate. The final product was suspended in water (3 mL), and methane sulfonic acid (0.035 mL) was added. Water was removed by lyophilization. M+1=478.2. ¹H NMR (CDCl₃) d (3H, 1.19 ppm), dd (1H, 2.73 ppm), dd (1H, 3.06 ppm), s (3H, 3.55 ppm), s (2H, 3.84 ppm), s (3H, 3.87 ppm), m (1H, 4.34 ppm), d (1H, 4.80 ppm), d (1H, 5.67 ppm), d (1H, 7.11 ppm), d (1H, 7.16 ppm), s (1H, 7.24 ppm), m (2H, 7.39 ppm), m (2H, 7.50 ppm), d (1H, 7.72 ppm), m (3H, 7.97 ppm).

### EXAMPLE 29

The following regioisomer was synthesized similar to that of N2-(1-(3-(aminomethyl)phenyl)propan-2-yl)-N-4-methyl-N-4-(3-methyl-7-phenyl-3H-benzo[d]imidazol-5-yl)pyrimidine-2,4-diamine: 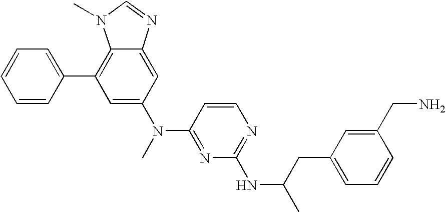
N2-(1-(3-(aminomethyl)phenyl)propan-2-yl)-N-4-methyl-N-4-(1-methyl-7-phenyl-1H-benzo[d]imidazol-5-yl)pyrimidine-2,4-diamine: M+1=478.1. ¹H NMR (CDCl₃) d (3H, 1.17 ppm), s (2H, 1.62 ppm), dd (1H, 2.71 ppm), dd (1H, 3.04 ppm), s (3H, 3.41 ppm), s (3H, 3.51 ppm), s (2H, 3.83 ppm), m (1H, 4.33 ppm), d (1H, 4.78 ppm), d (1H, 5.66 ppm), d (1H, 7.04 ppm), m (3H, 7.14 ppm) d (1H, 7.23 ppm), m (5H, 7.45 ppm), d (1H, 7.67 ppm), s (1H, 7.85 ppm).

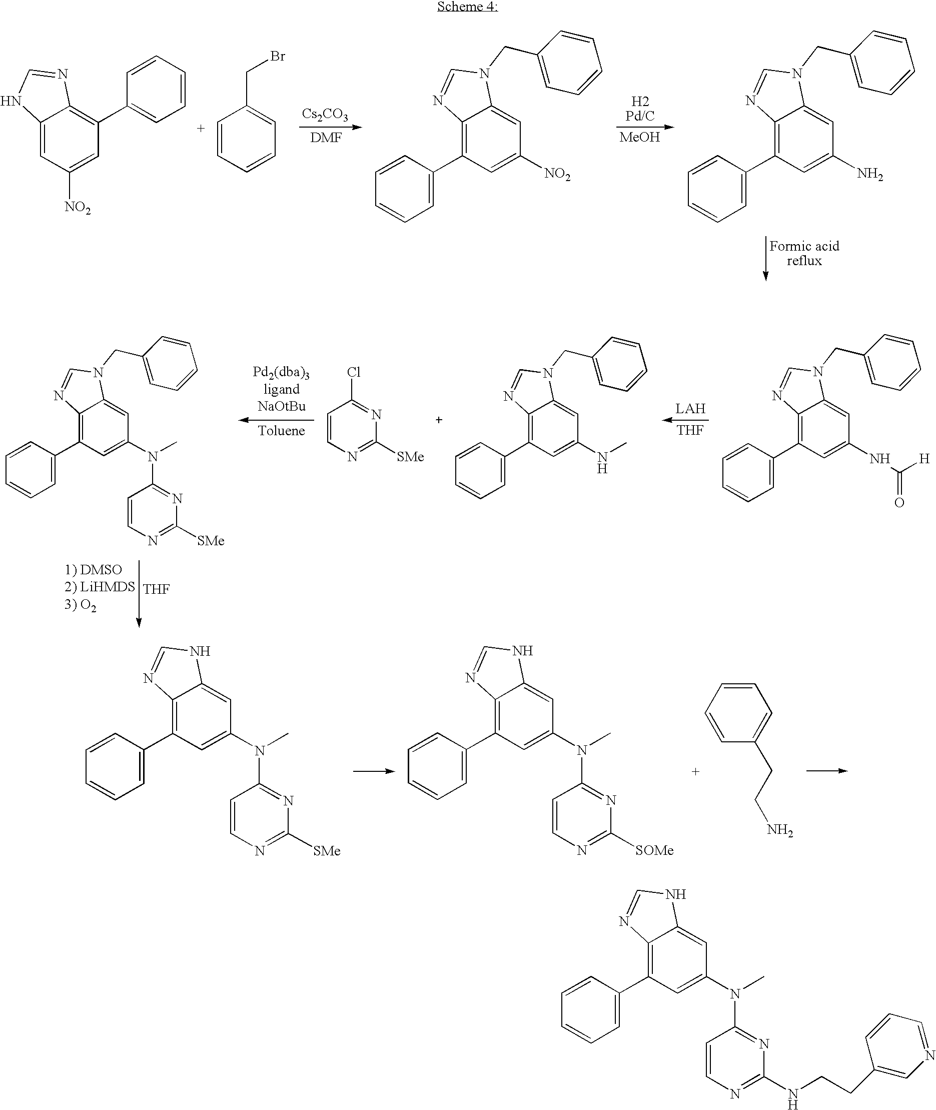

### EXAMPLE 30

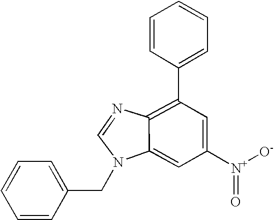 1-Benzyl-6-nitro-4-phenyl-1H-benzo[d]imidizole: To a stirring suspension of 6-nitro-4-phenyl-1H-benzo[d]imidizole (980 mg, 4.1 mmol) and cesium carbonate (1.6 g, 4.9 mmol) in DMF (15 mL) at 0° C. under nitrogen was added a solution of benzyl bromide (0.58 mL, 4.9 mmol) in DMF (5 mL). The ice bath was removed and the suspension was stirred for 3 h warming to room temperature. The solvent was removed under reduced pressure, and the resulting residue was partitioned between DCM (50 mL) and 5% NaHCO₃ (100 mL). The organic layer was washed twice with water (50 mL) and once with brine (20 mL). The solvent was removed under reduced pressure and the resulting solid was triturated with methanol. M+1=330.

### EXAMPLE 31

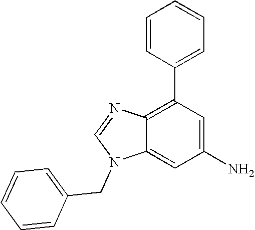 3-Benzyl-7-phenyl-3H-benzo[d]imidazol-5-amine: To a stirring suspension of 1-benzyl-6-nitro-4-phenyl-1H-benzo[d]imidizole (1.1 g, 3.3 mmol) in methanol (50 mL) under nitrogen was added palladium on carbon (10%, 100 mg). The suspension was stirred over an atmosphere of hydrogen for 1 h at room temperature. The reaction was filtered through a bed of Celite, and the filtrate was evaporated to dryness under reduced pressure. M+1=300.

### EXAMPLE 32

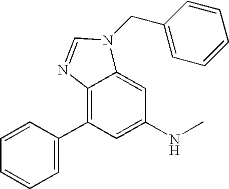 3-Benzyl-N-methyl-7-phenyl-3H-benzo[d]imidazol-5-amine: A suspension of 3-benzyl-7-phenyl-3H-benzo[d]imidazol-5-amine (0.80 g, 2.7 mmol) and formic acid (25 mL) was heated to reflux overnight. The solvent was removed under reduced pressure and the residue was suspended in THF (20 mL). The suspension was chilled to 0° C. and 1M lithium aluminum hydride in THF (4 mL) was added drop wise. The suspension was stirred for 1 h warming to 25° C. The reaction was quenched with solid sodium sulfate decahydrate. The salts were removed with filtration and the filtrate was concentrated to solids under reduced pressure. The resulting residue was purified on silica eluting with ammonia in methanol/DCM. M+1=314.

### EXAMPLE 33

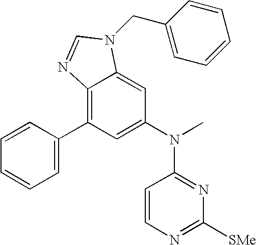 3-Benzyl-N-methyl-N-(2-(methylthio)pyrimidin-4-yl)-7-phenyl-3H-benzo [d]imidazol-5-amine: A 20 mL vessel was charged with 3-benzyl-N-methyl-7-phenyl-3H-benzo[d]imidazol-5-amine (490 mg, 1.6 mmol), 4-chloro-2-(methylthio)pyrimidine (0.27 mL, 2.3 mmol), 2-dicyclohexylphosphino-2′-(N,N-Dimethylamino)biphenyl (123 mg, 0.31 mmol), tris(dibenzylideneacetone) dipalladium (0) (145 mg, 0.16 mmol), sodium tert-butoxide (221 mg, 2.3 mmol), and toluene (15 mL). The vessel was capped and stirred at 110° C. for 18 h. The suspension was partitioned between DCM (50 mL) and 5% NaHCO₃ (25 mL). The organic layer was dried over MgSO₄. The product was purified by silica chromatography eluting with ammonia in methanol/DCM. M+1=428.

### EXAMPLE 34

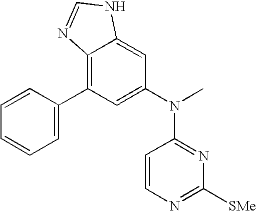 N-Methyl-N-(2-(methylthio)pyrimidin-4-yl)-7-phenyl-3H-benzo[d]imidazol-5-amine: To a stirring solution of 3-benzyl-N-methyl-N-(2-(methylthio)pyrimidin-4-yl)-7-phenyl-3H-benzo[d]imidazol-5-amine (120 mg, 0.27 mmol) and dimethyl sulfoxide (0.1 mL, 1.4 mmol) in THF (1.5 mL) was added lithium bis(trimethylsilyl) amide solution (1M in THF, 1.4 mL) at 25° C. The reaction was stirred for 1 h. The solvent was removed under reduced pressure and the residue was partitioned between DCM (10 mL) and 0.5M NaOH (5 mL). The aqueous layer was extracted twice with DCM (5 mL). The product was purified on silica eluting with ammonia in methanol/DCM. M+1=348.

### EXAMPLE 35

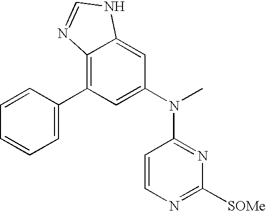 N-Methyl-N-(2-(methylsulfinyl)pyrimidin-4-yl)-7-phenyl-3H-benzo[d]imidazol-5-amine: To a stirring solution of N-methyl-N-(2-(methylthio)pyrimidin-4-yl)-7-phenyl-3H-benzo[d]imidazol-5-amine (80 mg, 0.23 mmol) in DCM (2 mL) was added 3-chloroperoxybenzoic acid (80 mg, 0.46 mmol). After 30 min, the reaction was diluted with DCM (10 mL), and washed with 1N NaOH (3 mL), then dried over MgSO₄. M+1=364/380.

### EXAMPLE 36

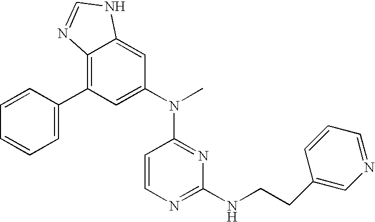 N4-Methyl-N-4-(7-phenyl-3H-benzo[d]imidazol-5-yl)-N-2-(2-(pyridin-3-yl)ethyl)-pyrimidine-2,4-diamine: In a 4 mL vessel was charged N-Methyl-N-(2-(methylsulfinyl)pyrimidin-4-yl)-7-phenyl-3H-benzo[d]imidazol-5-amine (80 mg, 0.23 mmol), 2-(pyridin-3-yl)ethanamine (0.1 mL, 0.82 mmol), and 1,4-dioxane (0.25 mL). The vessel was capped, then heated to 110° C. for 18 h. The product was purified on silica eluting with ammonia in methanol/DCM. M+1=422.3. ¹H NMR (CDCl₃) t (2H, 2.94 ppm), s (3H, 3.51 ppm), m (2H, 3.66 ppm), s (1H, 5.04 ppm), d (1H, 5.70 ppm), m (1H, 7.20 ppm), m (1H, 7.25 ppm), m (1H, 7.38 ppm), m (2H, 7.46 ppm), d (1H, 7.52 ppm), d (1H, 7.68 ppm), s (2H, 7.80 ppm), s (1H, 8.05 ppm), m (2H, 8.44 ppm).
Biological Assays

The following assays were used to characterize the ability of compounds of the invention to inhibit the production of TNF-α and IL-1-β. The second assay can be used to measure the inhibition of TNF-α and/or IL-1-β in mice after oral administration of the test compounds. The third assay, a glucagon binding inhibition in vitro assay, can be used to characterize the ability of compounds of the invention to inhibit glucagon binding. The fourth assay, a cyclooxygenase enzyme (COX-1 and COX-2) inhibition activity in vitro assay, can be used to characterize the ability of compounds of the invention to inhibit COX-1 and/or COX-2. The fifth assay, a Raf-kinase inhibition assay, can be used to characterize the compounds of the invention to inhibit phosphorylation of MEK by activated Raf-kinase.

Lipopolysaccharide-Activated Monocyte TNF Production Assay

Isolation of Monocytes

Test compounds were evaluated in vitro for the ability to inhibit the production of TNF by monocytes activated with bacterial lipopolysaccharide (LPS). Fresh residual source leukocytes (a byproduct of plateletpheresis) were obtained from a local blood bank, and peripheral blood mononuclear cells (PBMCs) were isolated by density gradient centrifugation on Ficol-Paque Plus (Pharmacia). PBMCs were suspended at 2×10⁶/mL in DMEM supplemented to contain 2% FCS, 10 mM, 0.3 mg/mL glutamate, 100 U/mL penicillin G and 100 mg/mL streptomycin sulfate (complete media). Cells were plated into Falcon flat bottom, 96 well culture plates (200 μL/well) and cultured overnight at 37° C. and 6% CO₂. Non-adherent cells were removed by washing with 200 μl/well of fresh medium. Wells containing adherent cells ([1 70% monocytes) were replenished with 100 μL of fresh medium.

Preparation of Test Compound Stock Solutions

Test compounds were dissolved in DMZ. Compound stock solutions were prepared to an initial concentration of 10-50 μM. Stocks were diluted initially to 20-200 μM in complete media. Nine two-fold serial dilutions of each compound were then prepared in complete medium.

Treatment of Cells with Test Compounds and Activation of TNF Production with Lipopolysaccharide

One hundred microliters of each test compound dilution were added to microtiter wells containing adherent monocytes and 100 μL complete medium. Monocytes were cultured with test compounds for 60 min at which time 25 μL of complete medium containing 30 ng/mL lipopolysaccharide from E. coli K532 were added to each well. Cells were cultured an additional 4 hrs. Culture supernatants were then removed and TNF presence in the supernatants was quantified using an ELISA.

TNF ELISA

Flat bottom, 96 well Corning High Binding ELISA plates were coated overnight (4° C.) with 150 μL/well of 3 μg/mL murine anti-human TNF-α MAb (R&D Systems #MAB210). Wells were then blocked for 1 h at RT with 200 μL/well of CaCl₂-free ELISA buffer supplemented to contain 20 mg/mL BSA (standard ELISA buffer: 20 mM, 150 mM NaCl, 2 mM CaCl₂, 0.15 mM thimerosal, pH 7.4). Plates were washed and replenished with 100 μL of test supernatants (diluted 1:3) or standards. Standards consisted of eleven 1.5-fold serial dilutions from a stock of 1 ng/mL recombinant human TNF (R&D Systems). Plates were incubated at RT for 1 h on orbital shaker (300 rpm), washed and replenished with 100 μL/well of 0.5 μg/mL goat anti-human TNF-α (R&D systems #AB-210-NA) biotinylated at a 4:1 ratio. Plates were incubated for 40 min, washed and replenished with 100 μL/well of alkaline phosphatase-conjugated streptavidin (Jackson ImmunoResearch #016-050-084) at 0.02 μg/mL. Plates were incubated 30 min, washed and replenished with 200 μL/well of 1 mg/mL of p-nitrophenyl phosphate. After 30 min, plates were read at 405 nm on a Vₘₐₓ plate reader.

Data Analysis

Standard curve data were fit to a second order polynomial and unknown TNF-α concentrations determined from their OD by solving this equation for concentration. TNF concentrations were then plotted vs. test compound concentration using a second order polynomial. This equation was then used to calculate the concentration of test compounds causing a 50% reduction in TNF production.

Compounds of the invention can also be shown to inhibit LPS-induced release of IL-1β, IL-6 and/or IL-8 from monocytes by measuring concentrations of IL-1β, IL-6 and/or IL-8 by methods well known to those skilled in the art. In a similar manner to the above described assay involving the LPS induced release of TNF-α from monocytes, compounds of this invention can also be shown to inhibit LPS induced release of IL-1β, IL-6 and/or IL-8 from monocytes by measuring concentrations of IL-1β, IL-6 and/or IL-8 by methods well known to those skilled in the art. Thus, the compounds of the invention may lower elevated levels of TNF-α, IL-1, IL-6, and IL-8 levels. Reducing elevated levels of these inflammatory cytokines to basal levels or below is favorable in controlling, slowing progression, and alleviating many disease states. All of the compounds are useful in the methods of treating disease states in which TNF-α, IL-1β, IL-6, and IL-8 play a role to the full extent of the definition of TNF-α-mediated diseases described herein.

Lipopolysaccharide-Activated THP1 Cell TNF Production Assay

THP1 cells are resuspended in fresh THP1 media (RPMI 1640, 10% heat-inactivated FBS, 1XPGS, 1XNEAA, plus 30 μM βME) at a concentration of 1E6/mL. One hundred microliters of cells per well are plated in a polystyrene 96-well tissue culture. One microgram per mL of bacterial LPS is prepared in THP1 media and is transferred to the wells. Test compounds are dissolved in 100% DMSO and are serially diluted 3 fold in a polypropylene 96-well microtiter plate (drug plate). HI control and LO control wells contain only DMSO. One microliter of test compound from the drug plate followed by 10 μL of LPS are transferred to the cell plate. The treated cells are induced to synthesize and secrete TNF-α at 37° C. for 3 h. Forty microliters of conditioned media are transferred to a 96-well polypropylene plate containing 110 μL of ECL buffer (50 mM Tris-HCl pH 8.0, 100 mM NaCl, 0.05% Tween 20, 0.05% NaN₃ and 1% FBS) supplemented with 0.44 nM MAB610 monoclonal Ab (R&D Systems), 0.34 nM ruthenylated AF210NA polyclonal Ab (R&D Systems) and 44 μg/mL sheep anti-mouse M280 Dynabeads (Dynal). After a 2 h incubation at RT with shaking, the reaction is read on the ECL M8 Instrument (IGEN Inc.). A low voltage is applied to the ruthenylated TNF-α immune complexes, which in the presence of TPA (the active component in Origlo), results in a cyclical redox reaction generating light at 620 nM. The amount of secreted TNF-α in the presence of compound compared with that in the presence of DMSO vehicle alone (HI control) is calculated using the formula:% control (POC)=(cpd−average LO)/(average HI−average LO)*100. Data (consisting of POC and inhibitor concentration in μM) is fitted to a 4-parameter equation (y=A+((B−A)/(1+((x/C)ˆD))), where A is the minimum y (POC) value, B is the maximum y (POC), C is the x (cpd concentration) at the point of inflection and D is the slope factor) using a Levenburg-Marquardt non-linear regression algorithm.

The following compounds exhibit activities in the THP1 cell assay (LPS induced TNF release) with IC₅₀ values of 20 μM or less:
(3-(2-(4-(methyl(3-methyl-7-phenyl-3H-benzo[d]imidazol-5-yl)amino)pyrimidin-2-ylamino)propyl)phenyl)methanol;
2-Amino-1-(4-((4-(methyl(3-methyl-7-phenyl-3H-benzo[d]imidazol-5-yl)amino)-pyrimidin-2-ylamino)methyl)piperidin-1-yl)ethanone;
N2-((1s,4s)-4-aminocyclohexyl)-N-4-methyl-N-4-(3-methyl-7-phenyl-3H-benzo[d]imidazol-5-yl)pyrimidine-2,4-diamine;
N2-((S)-1-(3-((S)-1-aminoethyl)phenyl)propan-2-yl)-N-4-methyl-N-4-(3-methyl-7-phenyl-3H-benzo[d]imidazol-5-yl)pyrimidine-2,4-diamine;
N2-(1-(3-(aminomethyl)phenyl)propan-2-yl)-N-4-methyl-N-4-(1-methyl-7-phenyl-1H-benzo [d] imidazol-5-yl)pyrimidine-2,4-diamine;
N2-(1-(3-(aminomethyl)phenyl)propan-2-yl)-N-4-methyl-N-4-(3-methyl-7-phenyl-3H-benzo[d]imidazol-5-yl)pyrimidine-2,4-diamine;
N2-(1-(3-(azidomethyl)phenyl)propan-2-yl)-N-4-methyl-N-4-(3-methyl-7-phenyl-3H-benzo[d]imidazol-5-yl)pyrimidine-2,4-diamine;
N2-(2-chlorophenethyl)-N-4-methyl-N-4-(3-methyl-7-phenyl-3H-benzo[d]imidazol-5-yl)pyrimidine-2,4-diamine;
N2-isobutyl-N-4-methyl-N-4-(3-methyl-7-phenyl-3H-benzo[d]imidazol-5-yl)-pyrimidine-2,4-diamine;
N4-methyl-N-4-(3-methyl-7-phenyl-3H-benzo[d]imidazol-5-yl)-N-2-(2-(pyridin-3-yl)ethyl)pyrimidine-2,4-diamine;
N4-methyl-N-4-(3-methyl-7-phenyl-3H-benzo[d]imidazol-5-yl)-N-2-(2-phenoxy-ethyl)pyrimidine-2,4-diamine;
N4-Methyl-N-4-(3-methyl-7-phenyl-3H-benzo[d] imidazol-5-yl)-N-2-(piperidin-4-ylmethyl)pyrimidine-2,4-diamine;
N4-Methyl-N-4-(7-phenyl-3H-benzo[d]imidazol-5-yl)-N-2-(2-(pyridin-3-yl)ethyl)-pyrimidine-2,4-diamine;
tert-Butyl 2-(4-((4-(methyl(3-methyl-7-phenyl-3H-benzo[d]imidazol-5-yl)amino)-pyrimidin-2-ylamino)methyl)piperidin-1-yl)-2-oxoethylcarbamate; and
tert-Butyl 4-(4-(methyl(3-methyl-7-phenyl-3H-benzo[d]imidazol-5-yl)amino)-pyrimidin-2-ylamino)piperidine-1-carboxylate.
   Inhibition of LPS-Induced TNF-α Production in Mice

Male DBA/1LACJ mice are dosed with vehicle or test compounds in a vehicle (the vehicle consisting of 0.5% tragacanth in 0.03 N HCl) 30 minutes prior to lipopolysaccharide (2 mg/Kg, I.V.) injection. Ninety minutes after LPS injection, blood is collected and the serum is analyzed by ELISA for TNF-α levels.

Compounds of the invention may be shown to have anti-inflammatory properties in animal models of inflammation, including carageenan paw edema, collagen induced arthritis and adjuvant arthritis, such as the carageenan paw edema model (C. A. Winter et al Proc. Soc. Exp. Biol. Med. (1962) vol 111, p 544; K. F. Swingle, in R. A. Scherrer and M. W. Whitehouse, Eds., Anti-inflammatory Agents, Chemistry and Pharmacology, Vol. 13-11, Academic, New York, 1974, p. 33) and collagen induced arthritis (D. E. Trentham et al J. Exp. Med. (1977) vol. 146, p 857; J. S. Courtenay, Nature (New Biol.) (1980), Vol 283, p 666).

¹²⁵I-Glucagon Binding Screen with CHO/hGLUR Cells

The assay is described in WO 97/16442, which is incorporated herein by reference in its entirety.

Reagents

The reagents can be prepared as follows: (a) prepare fresh 1M o-Phenanthroline (Aldrich) (198.2 mg/mL ethanol); (b) prepare fresh 0.5M DTT (Sigma); (c) Protease Inhibitor Mix (1000×): 5 mg leupeptin, 10 mg benzamidine, 40 mg bacitracin and 5 mg soybean trypsin inhibitor per mL DMSO and store aliquots at −20° C.; (d) 250 μM human glucagon (Peninsula): solubilize 0.5 mg vial in 575 μl 0.1N acetic acid (1 μL yields 1 μM final concentration in assay for non-specific binding) and store in aliquots at −20° C.; (e) Assay Buffer: 20 mM Tris (pH 7.8), 1 mM DTT and 3 mM o-phenanthroline; (f) Assay Buffer with 0.1% BSA (for dilution of label only; 0.01% final in assay): 10 μL 10% BSA (heat-inactivated) and 990 μL Assay Buffer; (g) ¹²⁵I-Glucagon (NEN, receptor-grade, 2200 Ci/mmol): dilute to 50,000 cpm/25 μL in assay buffer with BSA (about 50 pM final concentration in assay).

Harvesting of CHO/hGLUR Cells for Assay

1. Remove media from confluent flask then rinse once each with PBS (Ca, Mg-free) and Enzyme-free Dissociation Fluid (Specialty Media, Inc.).

2. Add 10 mL Enzyme-free Dissoc. Fluid and hold for about 4 min at 37° C.

3. Gently tap cells free, triturate, take aliquot for counting and centrifuge remainder for 5 min at 1000 rpm.

4. Resuspend pellet in Assay Buffer at 75000 cells per 100 μL.

Membrane preparations of CHO/hGLUR cells can be used in place of whole cells at the same assay volume. Final protein concentration of a membrane preparation is determined on a per batch basis.

Assay

The determination of inhibition of glucagon binding can be carried out by measuring the reduction of I¹²⁵-glucagon binding in the presence of compounds of Formula I. The reagents are combined as follows:

| | Compound/ | 250 μM | | CHO/hGLUR |
|---|---|---|---|---|
| | Vehicle | Glucagon | ¹²⁵I-Glucagon | Cells |
| Total | —/5 μl | — | 25 μL | 100 μL |
| Binding + | 5 μl/— | — | 25 μL | 100 μL |
| Compound | | | | |
| Nonspecific | —/5 μl | 1 μl | 25 μL | 100 μL |
| Binding | | | | |

The mixture is incubated for 60 min at 22° C. on a shaker at 275 rpm. The mixture is filtered over pre-soaked (0.5% polyethylimine (PEI)) GF/C filtermat using an Innotech Harvester or Tomtec Harvester with four washes of ice-cold 20 mM Tris buffer (pH 7.8). The radioactivity in the filters is determined by a gamma-scintillation counter.

Thus, compounds of the invention may also be shown to inhibit the binding of glucagon to glucagon receptors.

Cyclooxygenrase Enzyme Activity Assay

The human monocytic leukemia cell line, THP-1, differentiated by exposure to phorbol esters expresses only COX-1; the human osteosarcoma cell line 143B expresses predominantly COX-2. THP-1 cells are routinely cultured in RPMI complete media supplemented with 10% FBS and human osteosarcoma cells (HOSC) are cultured in minimal essential media supplemented with 10% fetal bovine serum (MEM-10% FBS); all cell incubations are at 37° C. in a humidified environment containing 5% CO₂.

COX-1 Assay

In preparation for the COX-1 assay, THP-1 cells are grown to confluency, split 1:3 into RPMI containing 2% FBS and 10 mM phorbol 12-myristate 13-acetate (TPA), and incubated for 48 h on a shaker to prevent attachment. Cells are pelleted and resuspended in Hank's Buffered Saline (HBS) at a concentration of 2.5×10⁶ cells/mL and plated in 96-well culture plates at a density of 5×10⁵ cells/mL. Test compounds are diluted in HBS and added to the desired final concentration and the cells are incubated for an additional 4 hours. Arachidonic acid is added to a final concentration of 30 mM, the cells incubated for 20 minutes at 37° C., and enzyme activity determined as described below.

COX-2 Assay

For the COX-2 assay, subconfluent HOSC are trypsinized and resuspended at 3×10⁶ cells/mL in MEM-FBS containing 1 ng human IL-1 b/mL, plated in 96-well tissue culture plates at a density of 3×10⁴ cells per well, incubated on a shaker for 1 hour to evenly distribute cells, followed by an additional 2 hour static incubation to allow attachment. The media is then replaced with MEM containing 2% FBS (MEM-2% FBS) and 1 ng human IL-1b/mL, and the cells incubated for 18-22 hours. Following replacement of media with 190 mL MEM, 10 mL of test compound diluted in HBS is added to achieve the desired concentration and the cells incubated for 4 hours. The supernatants are removed and replaced with MEM containing 30 mM arachidonic acid, the cells incubated for 20 minutes at 37° C., and enzyme activity determined as described below.

COX Activity Determined

After incubation with arachidonic acid, the reactions are stopped by the addition of 1N HCl, followed by neutralization with 1N NaOH and centrifugation to pellet cell debris. Cyclooxygenase enzyme activity in both HOSC and THP-1 cell supernatants is determined by measuring the concentration of PGE₂ using a commercially available ELISA (Neogen #404110). A standard curve of PGE₂ is used for calibration, and commercially available COX-1 and COX-2 inhibitors are included as standard controls.

Raf Kinase Assay

In vitro Raf kinase activity is measured by the extent of phosphorylation of the substrate MEK (Map kinase/ERK kinase) by activated Raf kinase, as described in GB 1,238,959 (incorporated herein by reference in its entirety). Phosphorylated MEK is trapped on a filter and incorporation of radiolabeled phosphate is quantified by scintillation counting.

Materials:

Activated Raf is produced by triple transfection of Sf9 cells with baculoviruses expressing “Glu-Glu”-epitope tagged Raf,val¹²-H-Ras, and Lck. The “Glu-Glu”-epitope, Glu-Try-Met-Pro-Met-Glu, was fused to the carboxy-terminus of full length c-Raf.

Catalytically inactive MEK (K97A mutation) is produced in Sf9 cells transfected with a baculovirus expressing c-terminus “Glu-Glu” epitope-tagged K97A MEK1.

Anti “Glu-Glu” antibody was purified from cells grown as described in: Grussenmeyer, et al., Proceedings of the National Academy of Science, U.S.A. pp 7952-7954, 1985.

Column buffer: 20 mM Tris pH 8, 100 mM NaCl, 1 mM EDTA, 2.5 mM EGTA, 10 mM MgCl₂, 2 mM DTT, 0.4 mM AEBSF, 0.1% n-octylglucopyranoside, 1 nM okadeic acid, and 10 μg/mL each of benzamidine, leupeptin, pepstatin, and aprotinin.

5× Reaction buffer: 125 mM HEPES pH=8, 25 mM MgCl₂, 5 mM EDTA, 5 mM Na₃VO₄, 100 μg/mL BSA.

Enzyme dilution buffer: 25 mM HEPES pH 8, 1 mM EDTA, 1 mM Na₃VO₄, 400 μg/mL BSA.

Stop solution: 100 mM EDTA, 80 mM sodium pyrophosphate.

Filter plates: Milipore multiscreen # SE3MO78E3, Immobilon-P (PVDF).

Methods:

Protein purification: Sf9 cells were infected with baculovirus and grown as described in Williams, et al., Proceedings of the National Academy of Science, U.S.A. pp 2922-2926, 1992. All subsequent steps were preformed on ice or at 4° C. Cells were pelleted and lysed by sonication in column buffer. Lysates were spun at 17,000×g for 20 min, followed by 0.22 μm filtration. Epitope tagged proteins were purified by chromatography over GammaBind Plus affinity column to which the “Glu-Glu” antibody was coupled. Proteins were loaded on the column followed by sequential washes with two column volumes of column buffer, and eluted with 50 μg/mL Glu-Tyr-Met-Pro-Met-Glu in column buffer.

Raf kinase assay: Test compounds were evaluated using ten 3-fold serial dilutions starting at 10-100 μM. 10 μL of the test inhibitor or control, dissolved in 10% DMSO, was added to the assay plate followed by the addition of 30 μL of the a mixture containing 10 μL 5× reaction buffer, 1 mM ³³P-γ-ATP (20 μCi/mL), 0.5 μL MEK (2.5 mg/mL), 1 μL 50 mM β-mercaptoethanol. The reaction was started by the addition of 10 μL of enzyme dilution buffer containing 1 mM DTT and an amount of activated Raf that produces linear kinetics over the reaction time course. The reaction was mixed and incubated at RT for 90 min and stopped by the addition of 50 μL stop solution. 90 μL aliquots of this stopped solution were transferred onto GFP-30 cellulose microtiter filter plates (Polyfiltronics), the filter plates washed in four well volumes of 5% phosphoric acid, allowed to dry, and then replenished with 25 μL scintillation cocktail. The plates were counted for ³³P gamma emission using a TopCount Scintillation Reader.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes, which are obvious to one skilled in the art, are intended to be within the scope and nature of the invention, which are defined, in the appended claims.

From the foregoing description, one skilled in the art can easily ascertain the essential characteristics of this invention, and without departing from the spirit and scope thereof, can make various changes and modifications of the invention to adapt it to various usages and conditions.

For the treatment of TNF-α, IL-1β, IL-6, and IL-8 mediated diseases, cancer, and/or hyperglycemia, the compounds of the present invention may be administered orally, parentally, by inhalation spray, rectally, or topically in dosage unit formulations containing conventional pharmaceutically acceptable carriers, adjuvants, and vehicles. The term “parenteral” as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition (also referred to as “medicament” herein) of either to a subject (i.e., an animal, preferably a mammal, most preferably a human) believed to be in need of preventative treatment, such as, for example, pain, inflammation and the like.

The dosage regimen for treating a TNF-α, IL-1, IL-6, and IL-8 mediated diseases, cancer, and/or hyperglycemia with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

What is claimed is:

1. A compound of the formula 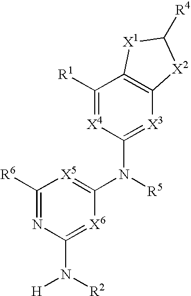
or a pharmaceutically acceptable salt or hydrate thereof, wherein
—X¹—CR⁴—X²— is selected from —N═CR⁴—NR^{a}—, —NR^{a}CR⁴═N—, —N═CR⁴—O—, —O—CR⁴═N—, —N═CR⁴—S—, —S—CR⁴═N—, —N═CR⁴—CR⁴— and —CR⁴—CR⁴═N—;
X³ is N or CR⁴;
X⁴ is N or CR⁴;
X⁵ is N or CR⁵;
X⁶ is N or CR⁶; wherein only 1, 2 or 3 of X¹, X², X³ and X⁴ are N;
R¹ is a saturated, partially saturated or unsaturated 5-, 6- or 7-membered, ring containing 0, 1, 2 or 3 atoms selected from N, O and S, wherein the ring is substituted by 0, 1, 2 or 3 substituents selected from C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, OR^{b}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O) R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};
R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R, —OC₂₋₆alkylOR^{a}, —SR, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, N(R^{a})C(═O)OR^{b}, N(R^{a})C(═O)NR^{a}R^{a}, N(R^{a})C(═NR^{a}) NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{g}, OC(O)N(R^{a})S(═O)₂R^{e}, —OC₂₋₆alkylNR^{a}R^{e}, —OC₂₋₆alkylOR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}; or
R² is a saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(=)OR^{b}, C(═O)NR^{a}R^{a}, C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})—C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})—NR^{a}R^{a}, —N(R^{a})S(═O)₂R^{b}, —N(R^{a})S(═O)₂NR^{a}R, —NR^{a}C₂₋₆alkylNR^{a}R^{g}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)—R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —C(═O)N(R^{a})S(═O)₂R^{e}, —OC₂₋₆alkylNR^{a}R^{e}, —OC₂₋₆alkylOR^{e}, —SW, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I;
R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})—C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})—S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};
R⁴ is independently in each instance H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, OR^{b}, OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, NR^{a}, —NR^{a}R^{a}, N(R^{a})C(═O)R^{b}, N(R^{a})C(═O)—OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)—₂NR^{a}R^{a}, —NR^{a}C₂₋₆alkylNR^{a}R^{a} or —NR^{a}C₂₋₆alkylOR^{a};
R⁵ is H, R^{e}, C₁₋₄haloalkyl, —C(═O)R^{b}, —C(═O)OR^{b}, C(═O)NR^{a}R^{a} or C(═NR^{a})NR^{a}R^{a};
R⁶ is independently in each instance H, C₁₋₈alkyl, C₁₋₄haloalkyl, —NR^{a}R^{a}, —OR^{a}, or halo;
R^{a} is independently, at each instance, H or R^{b};
R^{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;
R^{d} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};
R^{e} is independently at each instance C₁₋₆alkyl substituted by 0, 1, 2 or 3 substituents independently selected from R^{d} and additionally substituted by 0 or 1 substituents selected from R^{g}; and
R^{g} is independently at each instance a saturated, partially saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, wherein the carbon atoms of the ring are substituted by 0, 1 or 2 oxo groups and the ring is substituted by 0, 1, 2 or 3 substituents selected from R^{b}, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)—OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

2. The compound according to claim 1, wherein
R¹ is a ring selected from phenyl, pyridyl, pyrimidinyl, pyridazine, pyrazine, pyrazole, imidazole, triazole, thiophene, furan, thiazole and oxazole, wherein the ring is substituted by 0, 1, 2 or 3 substituents selected from C₁₋₄alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};
R² is C₂₋₈alkyl substituted by 1 or 2 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylOR^{a}, —C(═O)R^{g}, —C(═O)—OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR, —N(R^{a})—C(═O)NR^{a}R^{g}, —C(═O)R^{e}, —C(═O)OR^{e}, —C(═O)NR^{a}R^{e}, —C(═NR^{a})NR^{a}R^{e}, OR^{e}, —OC(═O)R^{e}, —OC(═O)NR^{a}R^{a}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)—R^{e}, —N(R^{a})C(═O)OR^{e}, —N(R^{a})C(═O)NR^{a}R^{e} and a ring selected from phenyl, pyridyl, pyrimidinyl, pyridazine, pyrazine, pyrazole, imidazole, triazole, thiophene, furan, thiazole and oxazole, wherein the ring is substituted by 0, 1 or 2 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, —OC(═O)R^{b}, —OC(═O)NR^{a}R^{a}, —N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})—C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I;
R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)—R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})—C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})—S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};
R⁴ is H, R^{d}, R^{e} or R^{g};
R⁵ is H, R^{e} or R^{g}; and
R⁶ is H.

3. The compound according to claim 1 that is selected from:
(3-(2-(4-(methyl(3-methyl-7-phenyl-3H-benzo[d]imidazol-5-yl)amino)pyrimidin-2-ylamino)propyl)phenyl)methanol;
2-Amino-1-(4-((4-(methyl(3-methyl-7-phenyl-3H-benzo[d]imidazol-5-yl)amino)-pyrimidin-2-ylamino)methyl)piperidin-1-yl)ethanone;
N2-((1 s,4s)-4-aminocyclohexyl)-N-4-methyl-N-4-(3-methyl-7-phenyl-3H-benzo[d]imidazol-5-yl)pyrimidine-2,4-diamine;
N2-((S)-1-(3-((S)-1-aminoethyl)phenyl)propan-2-yl)-N-4-methyl-N-4-(3-methyl-7-phenyl-3H-benzo[d]imidazol-5-yl)pyrimidine-2,4-diamine;
N2-(1-(3-(aminomethyl)phenyl)propan-2-yl)-N-4-methyl-N-4-(1-methyl-7-phenyl-1H-benzo[d]imidazol-5-yl)pyrimidine-2,4-diamine;
N2-(1-(3-(aminomethyl)phenyl)propan-2-yl)-N-4-methyl-N-4-(3-methyl-7-phenyl-3H-benzo[d]imidazol-5-yl)pyrimidine-2,4-diamine;
N-2-(1-(3-(azidomethyl)phenyl)propan-2-yl)-N-4-methyl-N-4-(3-methyl-7-phenyl-3H-benzo[d]imidazol-5-yl)pyrimidine-2,4-diamine;
N2-(2-chlorophenethyl)-N-4-methyl-N-4-(3-methyl-7-phenyl-3H-benzo[d]imidazol-5-yl)pyrimidine-2,4-diamine;
N2-isobutyl-N-4-methyl-N-4-(3-methyl-7-phenyl-3H-benzo[d]imidazol-5-yl)-pyrimidine-2,4-diamine;
N4-methyl-N-4-(3-methyl-7-phenyl-3H-benzo[d]imidazol-5-yl)-N-2-(2-(pyridin-3-yl)ethyl)pyrimidine-2,4-diamine;
N4-methyl-N-4-(3-methyl-7-phenyl-3H-benzo [d]imidazol-5-yl)-N-2-(2-phenoxy-ethyl)pyrimidine-2,4-diamine;
N4-Methyl-N-4-(3-methyl-7-phenyl-3H-benzo[d]imidazol-5-yl)-N-2-(piperidin-4-ylmethyl)pyrimidine-2,4-diamine;
N4-Methyl-N-4-(7-phenyl-3H-benzo[d]imidazol-5-yl)-N-2-(2-(pyridin-3-yl)ethyl)-pyrimidine-2,4-diamine;
tert-Butyl 2-(4-((4-(methyl(3-methyl-7-phenyl-3H-benzo[d]imidazol-5-yl)amino)-pyrimidin-2-ylamino)methyl)piperidin-1-yl)-2-oxoethylcarbamate; and
tert-Butyl 4-(4-(methyl(3-methyl-7-phenyl-3H-benzo[d]imidazol-5-yl)amino)-pyrimidin-2-ylamino)piperidine-1-carboxylate.
or a pharmaceutically-acceptable salt or hydrate thereof.

4. A pharmaceutical composition comprising a compound according to claim 1 and a pharmaceutically acceptable carrier.

5. A method of treatment of inflammation comprising administering an effective amount of a compound according to claim 1.

6. A method of treatment of rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount of a compound according to claim 1.

7. A method of lowering plasma concentrations of either or both TNF-α and IL-1 comprising administering an effective amount of a compound according to claim 1.

8. A method of lowering plasma concentrations of either or both IL-6 and IL-8 comprising administering an effective amount of a compound according to claim 1.

9. A method of treatment of diabetes disease in a mammal comprising administering an effective amount of a compound according to claim 1 to produce a glucagon antagonist effect.

10. A method of treatment of a pain disorder in a mammal comprising administering an effective amount of a compound according to claim 1.

11. A method of decreasing prostaglandins production in a mammal comprising administering an effective amount of a compound according to claim 1.

12. A method of decreasing cyclooxygenase enzyme activity in a mammal comprising administering an effective amount of a compound according to claim 1.
